(19) United States
(12) Patent Application Publication
(10) Pub. No.: US 2005/0234493 A1
(43) Pub. Date: Oct. 20, 2005

(54) Methods and devices for cavity creation in mammalian bone tissue

(76) Inventors: John P. Carr, River Vale, NJ (US); Julie N. Carr, River Vale, NJ (US); Matthew Kulik, Kinnelon, NJ (US)

Correspondence Address: FISH & RICHARDSON P.C., CITIGROUP CENTER 52ND FLOOR, 153 EAST 53RD STREET, NEW YORK, NY, 10022-4611, US

(21) Appl. No.: 11/094,620

(22) Filed: Mar. 30, 2005

Related U.S. Application Data
(60) Provisional application No. 60/558,330, filed on Mar. 31, 2004.
(60) Provisional application No. 60/558,860, filed on Apr. 2, 2004.

(51) Int. Cl. A61B 17/58

(52) U.S. Cl. 606/181

(57) ABSTRACT

A hollow sleeve has an open end. A guide rod is coupled to the hollow sleeve and extends axially from the open end. A bumper is coupled to a distal end of the guide rod. A deformable material is positionable between the open end and the bumper. A plunger is adapted to move axially through the hollow sleeve and apply a compressive force on the deformable material against a counterforce applied by the bumper.

### REFERENCE TO PRIOR APPLICATIONS

This application claims the benefit of U.S. Provisional Application No. 60/558,330, filed Mar. 31, 2004 and U.S. Provisional Application No. 60/558,860, filed Apr. 2, 2004.

### FIELD OF THE DISCLOSURE

This disclosure relates to methods and devices for cavity creation in mammalian bone tissue.

### BACKGROUND

Vertebral compression fractures and other bone damage may result from degenerative bone disease. When cancellous bone is diseased, the surrounding cortical bone may be more prone to compression fracture or collapse, because the cancellous bone no longer provides interior support for the surrounding bone.

A common bone disease that may lead to vertebral compression fractures and other bone damage is osteoporosis. Osteoporotic loss of calcium and phosphate salts in the bone may result in decreased bone mass and a loss of bone-structure strength.

Bone mass loss, decreased bone-structure strength, vertebral compression fractures and other bone damage also may be caused by a number of other diseases or conditions. For example, osteolysis, avascular necrosis, and bone cancer (e.g., osteolytic metastasis, myeloma, and malignant tumors of the bone marrow) predispose the bone to fracture or collapse. Other pathologies that may be related to such progressive bone diseases and vertebral compression fractures include posture change (slumping), decubitus ulcers (bed sores), hemangiomas, and neoplasms.

Vertebral compression fractures may cause the spine to compress resulting in kyphosis (round back) or scoliosis (spinal curvature). As the kyphosis increases, a person's head may migrate forward, causing chronic neck tension. Increased kyphosis also may limit one's ability to breathe freely. Vertebral compression fractures may impair a person's ability to walk unassisted; it may lead to a loss of height, severe back pain and deformity and cause prolonged or permanent disability or even death. Further, degenerative vertebral bone diseases may lead to the collapsing of the vertebra and the onset of painful bone-on-bone rubbing. Other types of bone damage may affect a patient in numerous ways.

Vertebroplasty is a conventional procedure that may be used to treat vertebral compression fractures. U.S. Pat. No. 6,273,916 (issued Aug. 14, 2001) and U.S. Pat. No. 6,048,346 (issued Apr. 11, 2000) relate to vertebroplasty. In vertebroplasty, bone cement may be used to reinforce a collapsed vertebra to provide increased structural support thereby relieving pain associated with undue pressure on the nerves.

A second conventional procedure is kyphoplasty, which is a technique that combines vertebroplasty with balloon catheter technology. U.S. Pat. No. 4,969,888 (issued Nov. 11, 1990); U.S. Pat. No. 5,108,404 (issued Apr. 28, 1992); U.S. Pat. No. 5,972,015 (issued Oct. 26, 1999); U.S. Pat. No. 6,066,154 (issued May 23, 2000); U.S. Pat. No. 6,235,043 (issued May 22, 2001); U.S. Pat. No. 6,423,083 (issued Jul. 23, 2002); U.S. Pat. No. 6,607,544 (issued Aug. 19, 2003); U.S. Pat. No. 6,623,505 (issued Sep. 23, 2003) and U.S. Pat. No. 6,663,647 (issued Dec. 16, 2003), relate to kyphoplasty.

### SUMMARY

Methods and devices for cavity creation in mammalian bone tissue are disclosed. In one embodiment, methods and devices are disclosed for cavity creation in weak, damaged, or diseased mammalian bone tissue, such as the cancellous bone tissue of mammalian vertebrae.

In another embodiment, methods and devices are disclosed for introducing an elastomeric material into bone tissue and applying a deforming force to the elastomeric material, whereupon the elastomeric material expands within the bone tissue to create a cavity. The deforming force applied to the elastomeric material is then removed thereby allowing the elastomeric material to resume its original shape. The elastomeric material is then withdrawn from the newly formed cavity.

The cavity so created can then be filled with a biocompatible filler, such as bone cement, using standard methods in the art. Preferably, the cavity is filled with bone cement using a Plexis™ or twistOR PrePack™ system, which are described in more detail below.

In some implementations, one or more of the following advantages may be present. The methods and devices disclosed may be useful to repair, stabilize, and reinforce weak, damaged, or diseased mammalian bone tissue and, therefore, may be useful to treat bone weakening and loss of bone mass, bone fracture, bone defects, and bone disease (such as, vertebral weakening and vertebral fractures, other bone damage and particularly vertebral compression fractures). Such conditions may have resulted from degenerative bone diseases, such as osteoporosis, osteolysis, avascular necrosis, bone cancer (e.g., osteolytic metastasis, myeloma, and malignant tumors of the bone marrow), and other diseases of bone that induce loss of bone mass or otherwise predispose the bone to weakening, fracture, or collapse.

### BRIEF DESCRIPTION OF THE FIGURES

These and other features, aspects, and advantages will become better understood with regard to the following description, examples, appended claims, and accompanying drawings where:

FIG. 1 is a perspective view of a cavity creation assembly depicting the elastomer exposed outside of the assembly with the plunger in an uncompressed position;

FIG. 2 is a perspective view of a cavity creation assembly with the plunger in an uncompressed position;

FIG. 3 is a perspective view of one embodiment of a cavity creation assembly with the plunger in a compressed position;

FIG. 4 is a perspective view of a plunger for use with a cavity creation device;

FIG. 5 is a perspective view of an elastomer holding sleeve for use with a cavity creation device;

FIG. 6 is a perspective view of an elastomer holding sleeve depicting the elastomer exposed outside the elastomer holding sleeve;

FIG. 7 is an expanded perspective view, partially broken away, depicting the elastomer holding sleeve;

FIGS. 8A and 9A are side views of a cavity creation assembly with the plunger in an uncompressed position;

FIGS. 8B and 9B are section views of a cavity creation assembly taken along line C-C of FIGS. 8A and 9A, respectively;

FIGS. 8C and 9C are exploded views of detail D of FIGS. 8B and 9B, respectively;

FIGS. 8D and 9D are a cross-sectional views of a cavity creation assembly taken from section E-E of FIGS. 8A and 9A depicting the guide rod support;

FIG. 10A is a side view of a cavity creation assembly prior to loading the elastomer with the plunger in a compressed position;

FIG. 10B is a section view of a cavity creation assembly taken along line A-A of FIG. 10A;

FIG. 10C is an exploded view of detail B of FIG. 10B;

FIG. 11A is a side view of a cavity creation assembly loaded with the elastomer, the plunger in a compressed position and the elastomer expanded;

FIG. 11B is a section view of the cavity creation assembly taken along line A-A of FIG. 1A;

FIG. 11C is an exploded view of detail B of FIG. 11B;

FIG. 12 is a perspective view of a cavity creation device;

FIG. 13 is a perspective view of a cavity creation device depicting the elastomer partially extending from the elastomer holding sleeve;

FIG. 14 is a perspective view of a cavity creation assembly following compression of the plunger and expansion of the elastomer;

FIG. 15 is a perspective view of a cavity creation assembly with the plunger having a ratcheted crank handle;

FIG. 16 is a cross-sectional view of a cavity creation assembly comprising a crank handle for operation with a threaded plunger and threaded elastomer holding sleeve;

FIG. 17 is a perspective view of a collapsible segmented elastomeric tube for use in a cavity creation device;

FIG. 18A is a perspective component view of a cavity creation assembly for use with a collapsible segmented elastomeric tube;

FIG. 18B is a perspective view of the holding sleeve of FIG. 18A;

FIG. 19 is a detailed drawing view of a cavity creation device for use with a collapsible segmented tube;

FIG. 20 is a perspective view of a delivery tube device;

FIG. 21 is an exploded perspective view, partially broken away, depicting the twistOR PrePack™ system;

FIG. 22 is a side elevation view, partially broken away and partially in cross-section depicting the twistOR PrePack™ system of FIG. 21 fully assembled;

FIG. 23 is an exploded perspective view of the transfer assembly member of the twistOR PrePack™ system;

FIG. 24 is a top plan view of the transfer assembly of FIG. 23;

FIG. 25 is a cross-sectional side elevation view of the transfer assembly taken along the line A-A of FIG. 24;

FIG. 26 is a side elevation view of the fully assembled twistOR PrePack™ system, partially broken away and partially in cross-section;

FIG. 27 is an enlarged cross-sectional side elevation view detailing area 7 of FIG. 26;

FIG. 28 is an exploded perspective view of an alternate embodiment of twistOR PrePack™;

FIG. 29 is a cross-sectional side elevation view of the twistOR PrePack™ of FIG. 28;

FIG. 30 is a cross-sectional side elevation view of the housing forming the twistOR PrePack™ transfer assembly of FIG. 28;

FIG. 31 is a top plan view of the twistOR PrePack™ housing of FIG. 30;

FIG. 32 is a perspective view of a fully assembled Plexis™ system;

FIGS. 33 and 34 are exploded and cross-section side elevation views of a Plexis™ system depicting the interrelation of component parts;

FIG. 35 is a cross-sectional view of a Plexis™ system in the mixing stage;

FIG. 36 is a cross-sectional view of a Plexis™ system depicting the mixed components transferring to the dispensing chamber;

FIGS. 37 and 38 are cross-sectional views of a Plexis™ system depicting the mixed components being dispensed from the dispensing chamber;

FIGS. 39A and 39B are perspective views of an exemplary manually actuable Plexis™ system in condition for mixing and dispensing, respectively; and

FIG. 40 is an exploded view of the exemplary manually actuable Plexis™ system.

### DETAILED DESCRIPTION

1.0 Devices

According to one implementation, a cavity creation device introduces an elastomer into bone tissue, such as a vertebral body, applies deforming forces to the elastomer to cause the elastomer to expand in the tissue to create a cavity. Upon removal of the deforming force, the elastomer withdraws back into the device leaving an empty cavity for subsequent filling with bone cement.

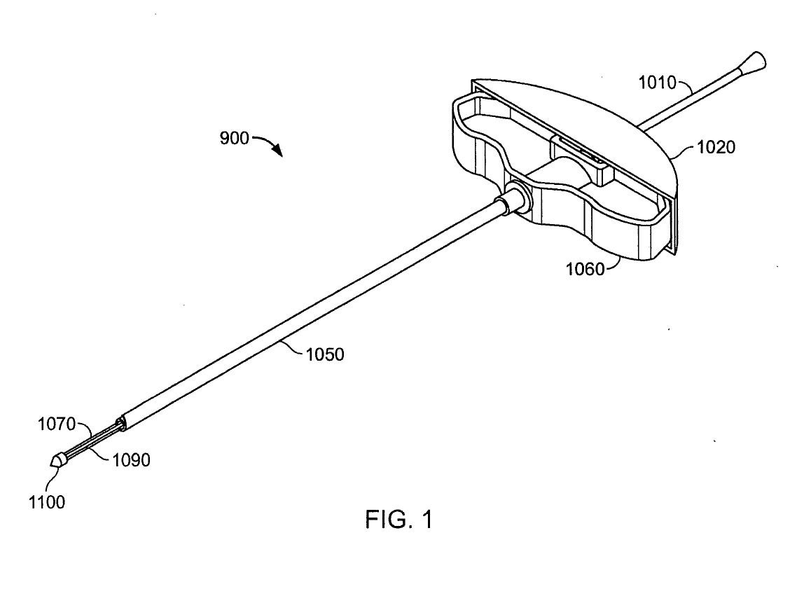

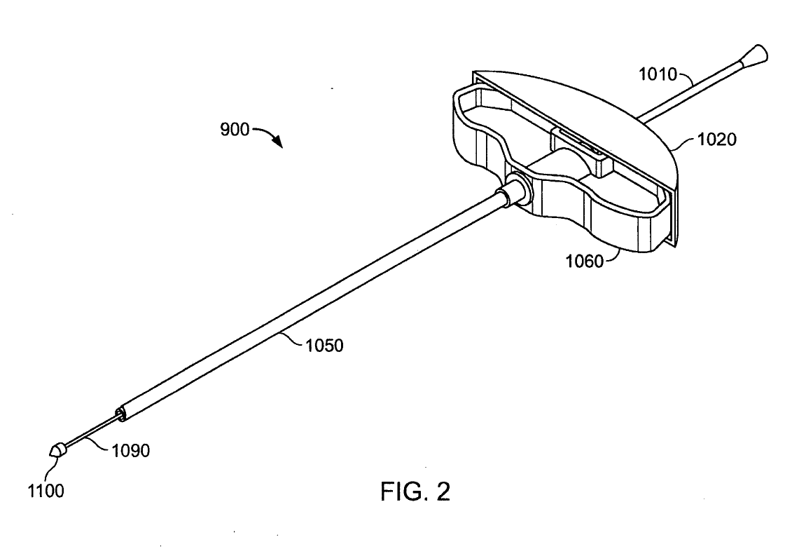

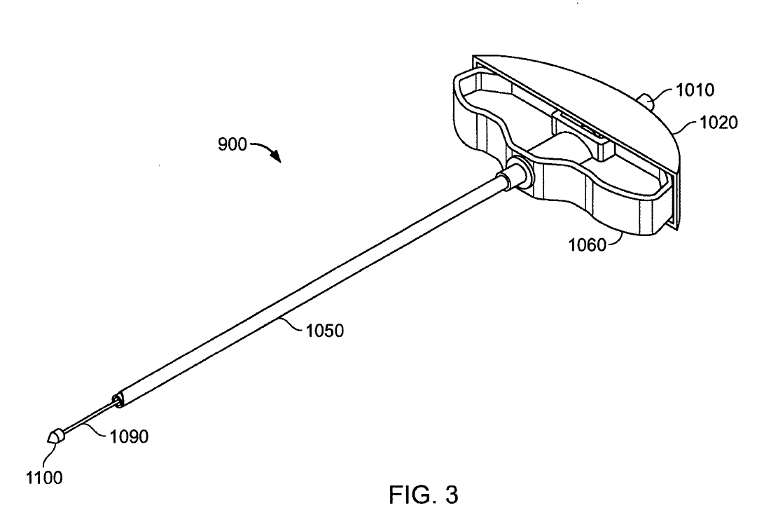

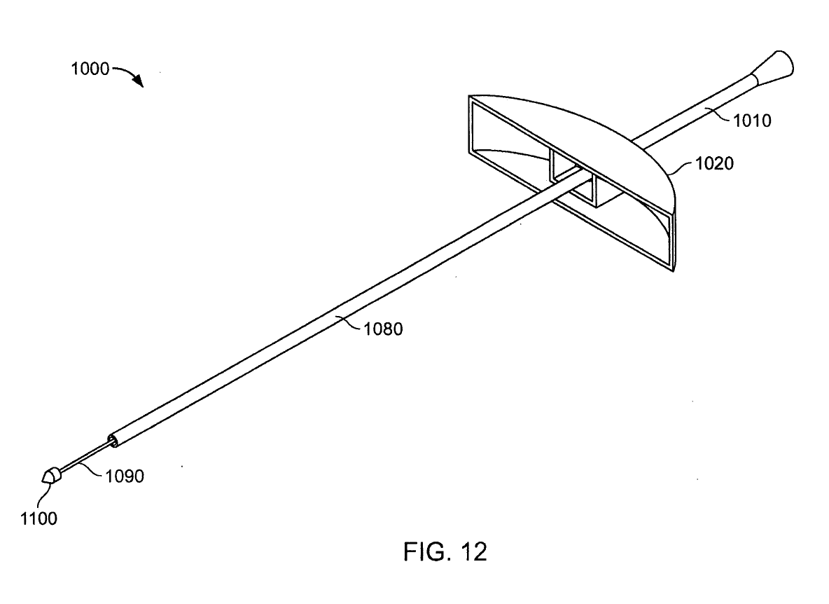

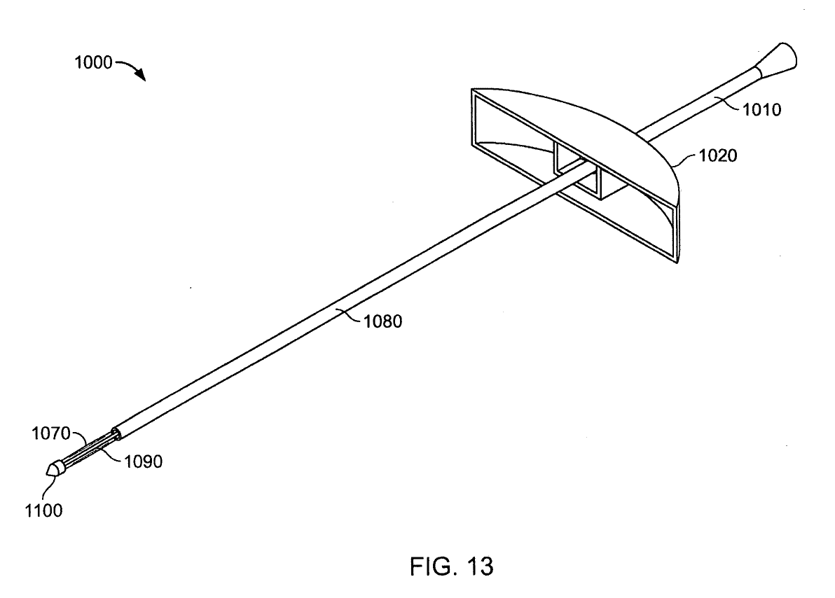

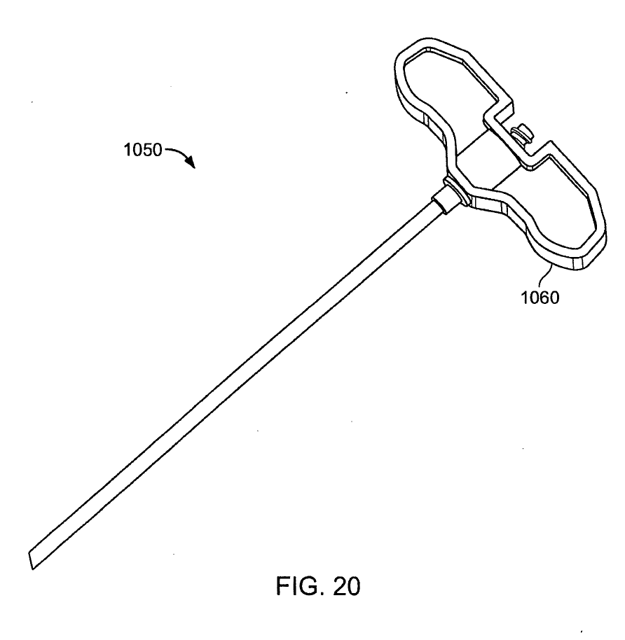

FIGS. 1-3 depict a particular embodiment of a cavity creation assembly 900, which comprises cavity creation device 1000 and delivery tube device 1050, such as a trocar. See FIGS. 12 and 13 for an illustration of particular cavity creation device 1000 by itself. And see FIG. 20 for an illustration of a particular delivery tube device 1050 by itself.

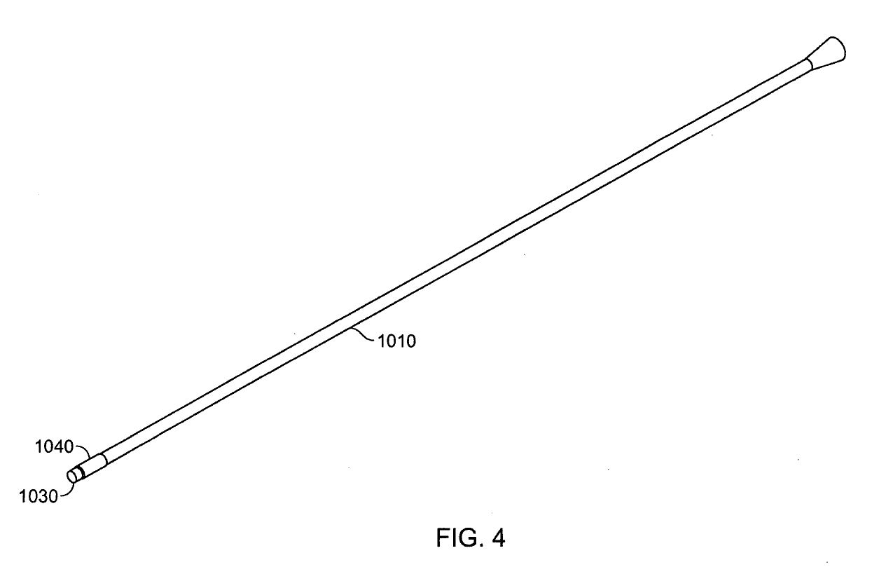

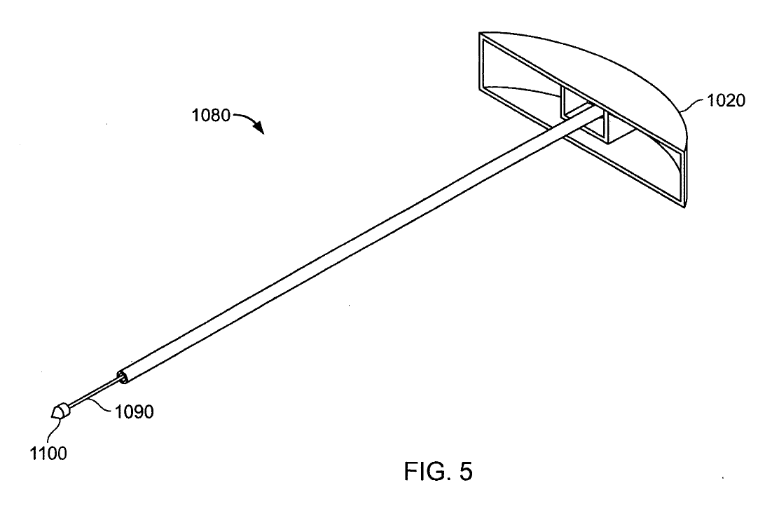

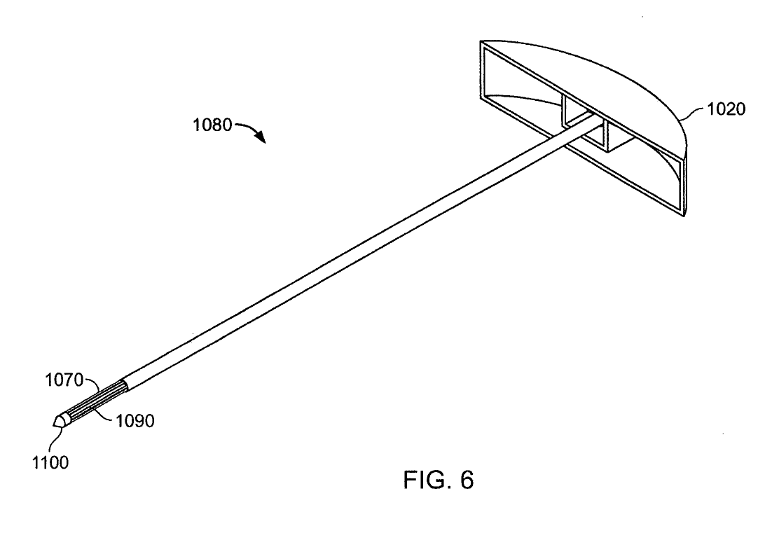

Referring to FIGS. 12 and 13, cavity creation device 1000 is depicted as comprising elastomer holding sleeve 1080 (see FIGS. 5 and 6), plunger 1010 (see FIG. 4) slid into elastomer holding sleeve 1080 and elastomer 1070 loaded at least partially within elastomer holding sleeve 1080. Preferably, elastomer 1070 is loaded in elastomer holding sleeve 1080 such that elastomer 1070 is adjacent to plunger head 1030 within elastomer holding sleeve 1080 or separated from plunger head 1030 by an air baffle. Elastomer holding sleeve 1080 and plunger 1010 can be constructed from any suitable materials, such as titanium, steel, or plastics.

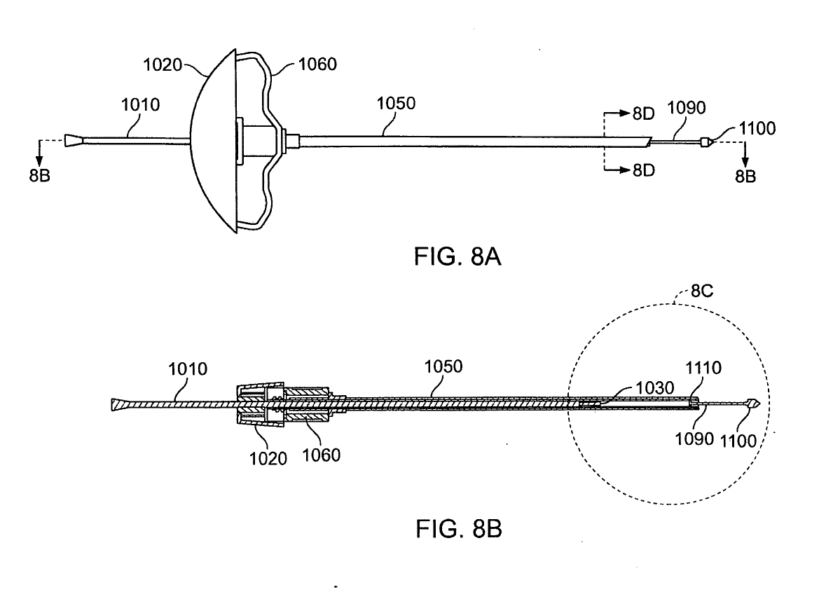

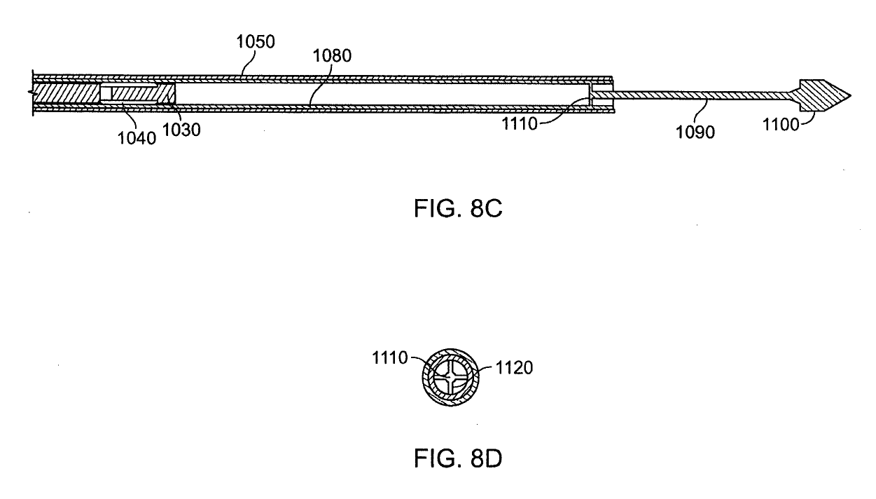

The illustrated cavity creation device 1000 is adapted to be inserted into delivery tube device 1050, wherein delivery tube device 1050 is first introduced into the vertebral body by any method well-known by those having ordinary skill in the art. Once delivery tube device 1050 is appropriately positioned within the vertebral body, cavity creation device 1000 is slid into delivery tube device 1050 such that the portion of elastomer holding sleeve 1080 having guide rod 1090 extending from sleeve 1080 is exposed from the end of delivery tube device 1050, as shown in FIGS. 1-3. Referring to FIGS. 8A and 8D, guide rod 1090 is fixed within elastomer holding sleeve 1080 by guide rod support 1110. Guide rod support 1110 is fused to the end of guide rod 1090 and includes a plurality of spokes 1120 spaced apart to allow passage of elastomer 1070.

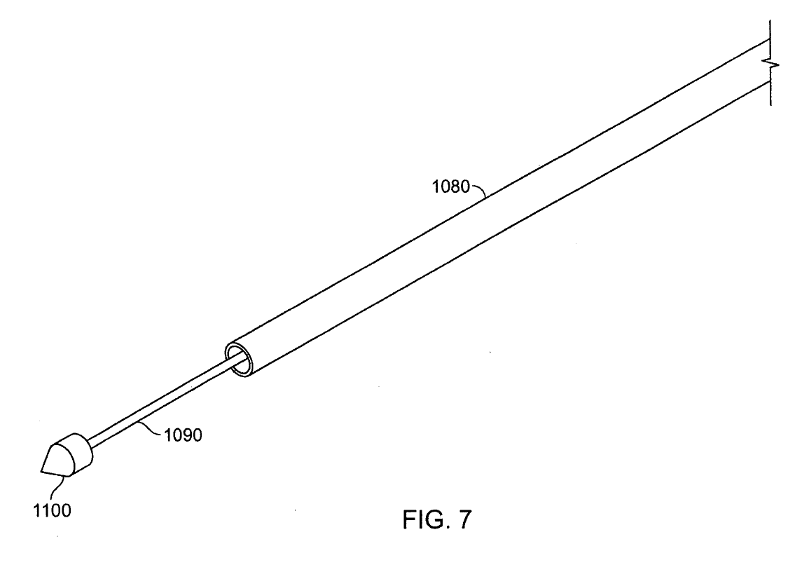

As shown in FIG. 7, guide rod 1090 includes bumper 1100 fixed at the end of guide rod 1090 extending from elastomer holding sleeve 1080. Preferably, bumper 1100 is pointed to allow for easier movement through the spongy bone of the vertebral body.

When elastomer holding sleeve 1080 and plunger 1010 are completely engaged through delivery tube device 1050, as in FIGS. 1-3, elastomer delivery and cavity creation can begin. Preferably, elastomer holding sleeve 1080 includes elastomer holding sleeve handle 1020 adapted to fit with delivery tube device handle 1060 to ensure proper assembly of cavity creation device 1000 and delivery tube device 1050.

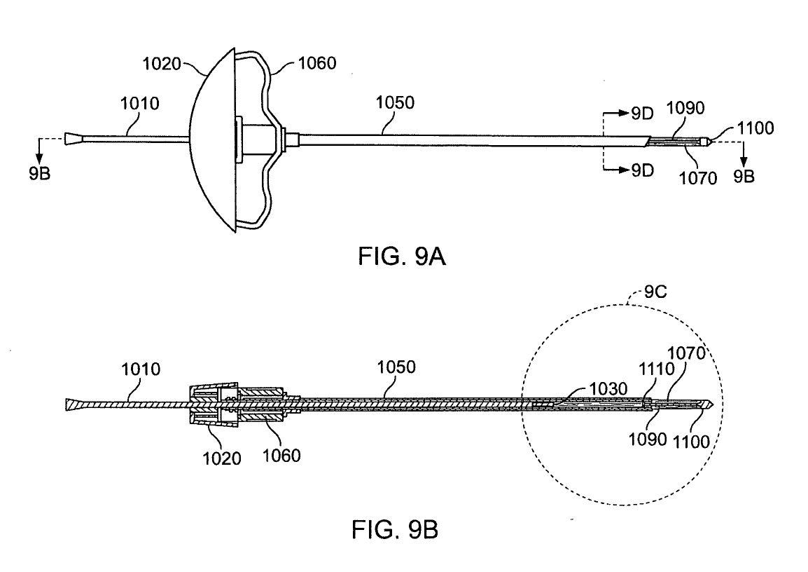

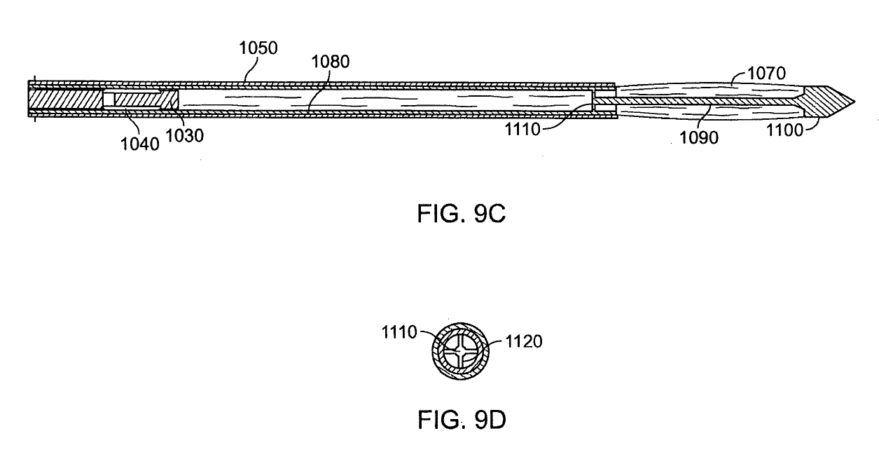

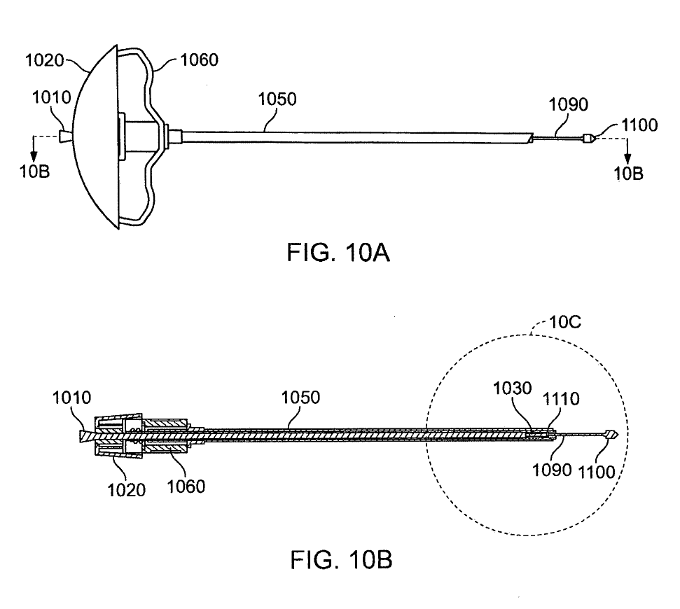

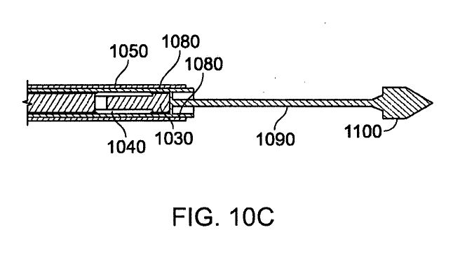

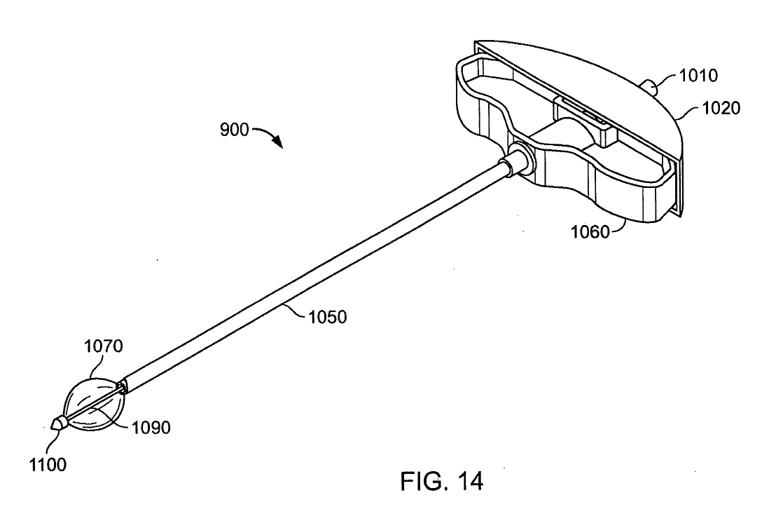

To initiate elastomer expansion and cavity creation, plunger 1010 must be compressed and driven through elastomer holding sleeve 1080 to apply a deforming force to elastomer 1070. One embodiment of plunger 1010 includes a simple manual pushing mechanism to compress plunger 1010 through elastomer holding sleeve 1080 and deliver the force upon elastomer 1070. In this embodiment, as shown in FIGS. 1-4 and 8-14, plunger 1010 has a syringe-like top which is manually pushed down to drive plunger head 1030 into contact with elastomer 1070. Alternatively, plunger head 1030 may be driven into an air baffle thereby creating an increased pressure acting on elastomer 1070. Release of the manual pressure placed on plunger 1010 will remove the force acting on elastomer 1070. Withdrawal of the plunger may allow the elastomer 1070 to return to an uncompressed position.

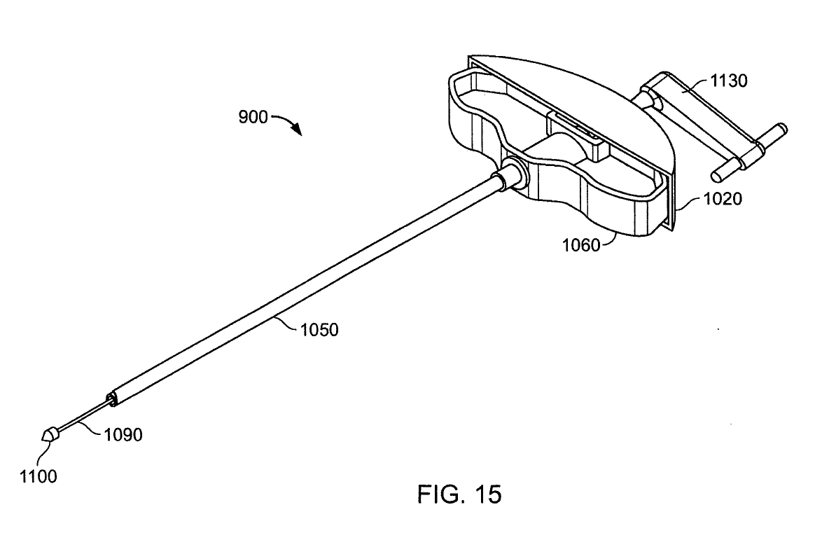

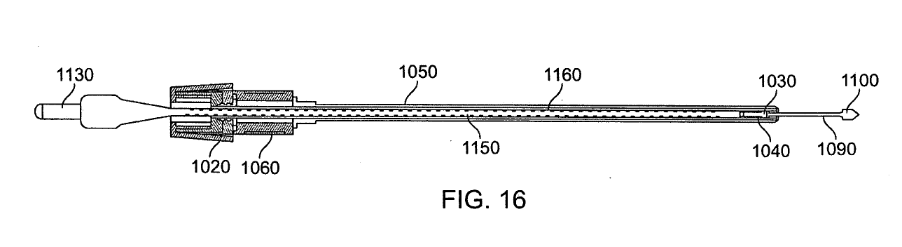

FIGS. 15 and 16 depict another embodiment of a plunger wherein threaded plunger 1150 is grooved within threaded elastomer holding sleeve 1160. Threaded plunger 1150 includes ratcheted crank handle 1130 for controlling the movement of threaded plunger 1150. Ratcheted crank handle 1130 may be turned to provide a force driving threaded plunger 1150 through threaded elastomer holding sleeve 1160 and into contact with elastomer 1070.

Alternatively, the head of threaded plunger 1150 may be driven into an air baffle thereby creating an increased pressure acting on elastomer 1070. The ratcheted crank handle 1130 provides a precise and quantified delivery of plunger force onto elastomer 1070. Removal of the plunger force is achieved by applying a reverse action to ratcheted crank handle 1130.

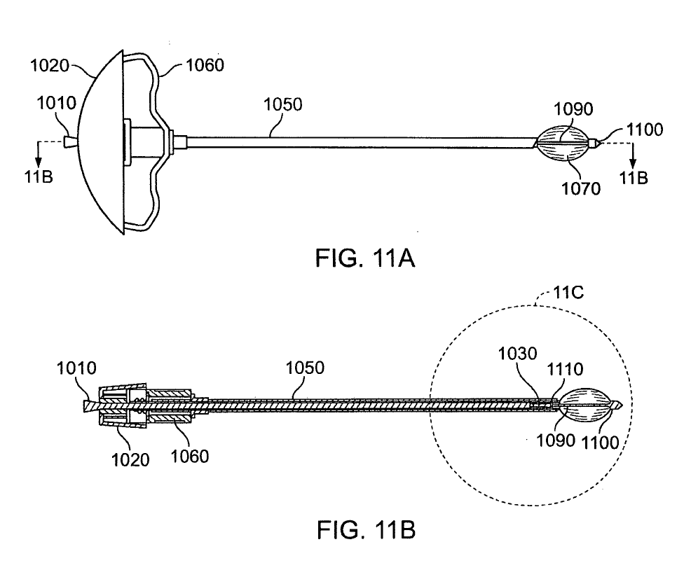

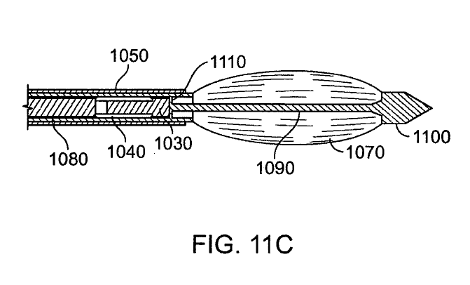

When the force is applied to elastomer 1070 by plunger 1010 (or threaded plunger 1150), elastomer 1070 will begin to expand in the path of least resistance, or toward bumper 1100. Bumper 1100, which maintains a fixed position at the end of guide rod 1090, provides a resistive force to elastomer 1070 when sufficient plunger force is applied to elastomer 1070 to cause elastomer 1070 to make contact with bumper 1100. As depicted in FIGS. 11A-1 IC and 14, in response to the resistive force from bumper 1100, elastomer 1070 will begin to expand in an outward direction relative to guide rod 1090. The expanded elastomer 1070 permeates the vertebral body and pushes against the cancellous bone to create a cavity in the vertebral body.

When the physician reaches the desired cavity size by means of mechanical (number of revolutions or distance compressed) and visual (CT scan) determinations, he or she can cease plunger movement and begin to back the plunger off elastomer 1070. Withdrawal of the plunger force will allow elastomer 1070 to move toward the area of least pressure or resistance, which will draw elastomer 1070 back into elastomer holding sleeve 1080. Once elastomer 1070 is completely retracted back into elastomer holding sleeve 1080, cavity creation device 1000 may be removed from delivery tube device 1050 allowing for introduction of a cement mixture into the created cavity via delivery tube device 1050.

1.1 Substitution Of Elastomer with a Collapsible Segmented Elastomeric Tube

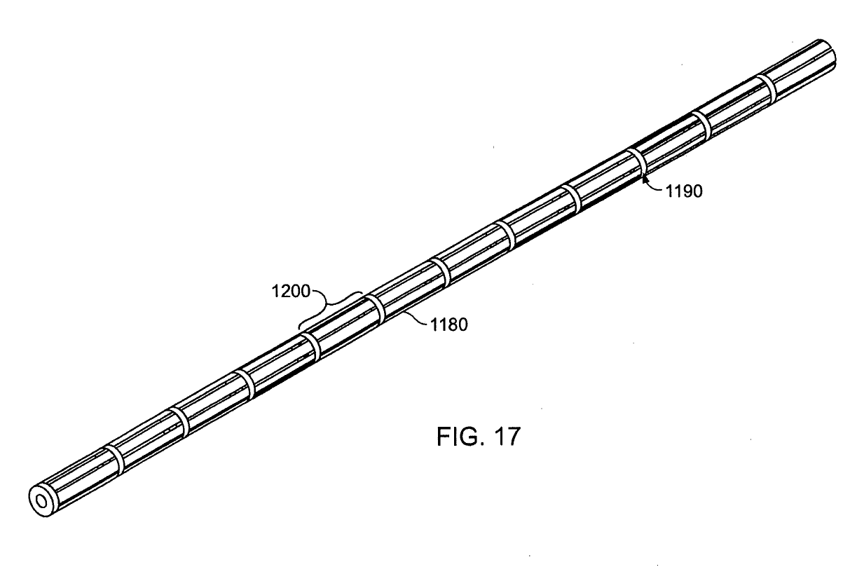

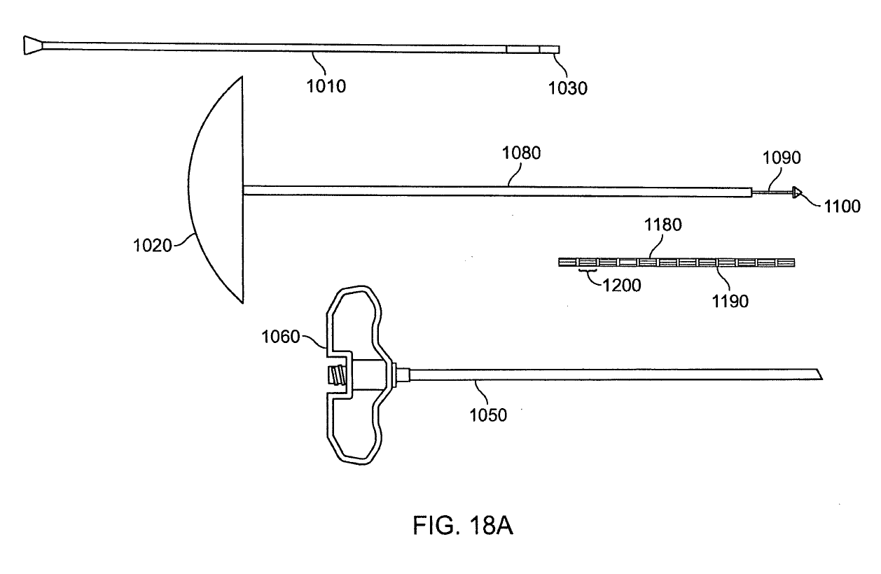

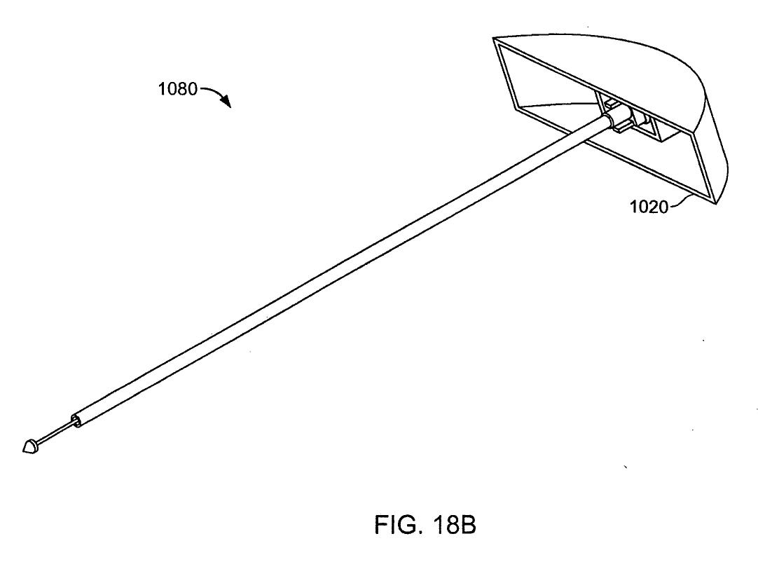

FIGS. 17-19 illustrate an additional embodiment, wherein elastomer 1070 is replaced with fused, collapsible segmented elastomeric tube 1180. As shown in FIGS. 17-19, collapsible segmented elastomeric tube 1180 is positioned over guide rod 1090 of elastomer holding sleeve 1080. Guide rod 1090 supports collapsible segmented elastomeric tube 1180 and provides a path for collapsible segmented elastomeric tube 1180 to follow when a compressive force is applied. The bottom of collapsible segmented elastomeric tube 1180 is fused to bumper 1100 at the end of elastomer holding sleeve 1080 and the top of collapsible segmented elastomeric tube 1180 is fused to the end of plunger 1010.

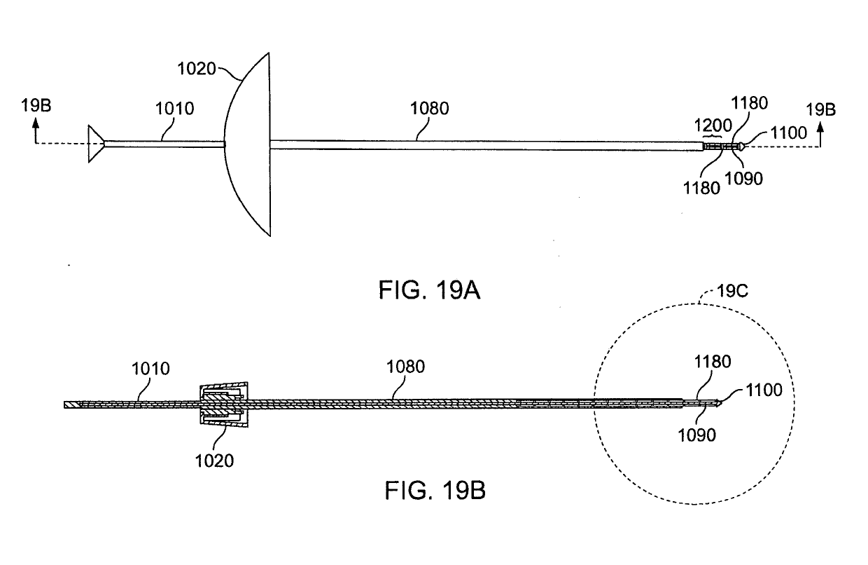

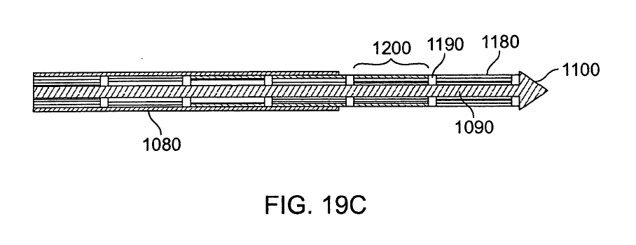

One to many elastomer segments 1200 of the collapsible segmented elastomeric tube 1180 are exposed at the end of the elastomer holding sleeve 1080 of cavity creation device 1000, as shown in FIG. 19. Collapsible segmented elastomeric tube 1180 is divided into various elastomer segments 1200 that are separated by support washers 1190 to which elastomer segments 1200 are chemically fused. Collapsible segmented elastomeric tube 1180 and plunger 1010 are bundled in the elastomer holding sleeve 1080 prior to use and does not require assembly in the field.

To create the cavity in the vertebral body, cavity creation device 1000 according to this embodiment is inserted through delivery tube device 1050, previously introduced into the vertebral body. The bottom end of cavity creation device 1000 protrudes past the bottom of delivery tube device 1050. Once cavity creation device 1000 is fully inserted into delivery tube device 1050, plunger 1010 is driven through elastomer holding sleeve 1080 by pushed manual force or by force generated by ratcheted crank handle 1130, as described in detail above. As plunger 1010 is driven through elastomer holding sleeve 1080, the elastomer segment 1200 located at the bottom of elastomer holding sleeve 1080 will collapse upon itself and spread outward in a radial direction. This may occur as the result of the elastomer segment 1200 being forced against a counterforce being applied by the bumper. As continued pressure is applied to plunger 1010, the other elastomer segments 1200 of collapsible segmented elastomeric tube 1180 will collapse on to one another in sequential order until all of the elastomer segments 1200 have collapsed. The elastomer segments 1200 spread within the vertebral body and push against the cancellous bone within the vertebral body thereby creating a cavity therein.

Once the desired cavity is created in the vertebral body, collapsible segmented elastomeric tube 1180 is removed from the newly formed cavity by retracting plunger 1010 out of elastomer holding sleeve 1080. Upon removal of the force applied by plunger 1010, the elastomer segments 1200 will retract to their original size and shape and slide back into their original alignment. When plunger 1010 is fully retracted within elastomer holding sleeve 1080, the entire cavity creation device 1000 is removed from delivery tube device 1050.

1.2 Bone Cavity Formation by Substituting Elastomer with a Primary Elastomer Encapsulated within a Secondary Casing

In another embodiment, elastomer 1070 (see FIG. 1) is encapsulated within a secondary casing prior to cavity creation. Preferably, the secondary casing is constructed of an elastomer or other expandable material, such as metal mesh. The secondary casing can be any biocompatible material that can expand, but need not have elastomeric properties. Referring to FIG. 1, this embodiment can be practiced by wrapping the expandable material (referred to herein as a “secondary casing”) around the open space between elastomer holding sleeve 1080 and bumper 1100. The secondary casing is mechanically attached to both elastomer holding sleeve 1080 and bumper 1100, defining an enclosed cylindrical space therebetween.

Plunger 1010 is inserted into elastomer holding sleeve 1080, whereupon primary elastomer 1070 is adjacent to plunger head 1030 or separated from the plunger head by an air baffle. When elastomer holding sleeve 1080 and plunger 1010 are completely engaged through delivery tube device 1050, the secondary casing region (which defines the enclosed cylindrical space that is located between elastomer holding sleeve 1080 and bumper 1100) is exposed, and the device is ready for use.

When used to treat a vertebral fracture, delivery tube device 1050 is inserted in the patient's vertebrae and the cavity creation device is pushed into the cancellous bone inside the patient's vertebral body. The secondary casing region extends within the bone tissue. Bumper 1100 is pointed to allow for easier movement through the spongy bone.

In operation, plunger 1010 forces primary elastomer 1070 toward bumper 1100 into the cylindrical space defined by the secondary casing. Plunger 1010 can be operated in one of two ways; a simple manual pushing or through ratcheted crank handle 1130 that utilizes a threaded plunger to deliver a more precise and quantified pressure. As plunger 1010 compresses elastomer 1070 against bumper 1100, elastomer 1070 expands in the path of least resistance, which is the region of the secondary casing. Bumper 1100, at the end of elastomer holding sleeve 1080, is immovable and acts to force elastomer 1070 to expand outward, in a direction perpendicular to guide rod 1090 within the secondary casing, causing the secondary casing to expand which forces bone tissue outward thereby forming a cavity within the vertebral body.

When the surgeon reaches the desired cavity size by means of mechanical (for example, the number of revolutions of delivery tube device handle 1060 or distance compressed) and/or visual (CT scan) determinations, he or she withdraws plunger 1010 through elastomer holding sleeve 1080. Upon withdrawal of plunger 1010, elastomer 1070 completely retracts within elastomer holding sleeve 1080. Cavity creation device 1000 can then be removed and bone cement can be introduced into the created cavity.

1.3 Bone Cavity Formation by Way of an Implantable Elastomeric Casing Expanded with Bone Cement or other Biocompatible Filler

In another embodiment, bone fractures, such as vertebral fractures can be treated by inserting a biocompatible, elastomeric casing into bone tissue and forcing a biocompatible filler, such as polymethylmethacrylate (PMMA) bone cement, into the casing whereby the casing expands within the bone tissue to correct and reinforce the bone structure. The expanded casing and biocompatible filler are left within the vertebral body as an implant. Preferably, the elastomer casing is chemically resistant to the biocompatible filler. It also may be preferable that the elastomer casing is approved by the Food and Drug Administration (FDA) for implantation within the human body.

Referring to FIG. 1, this embodiment can be practiced by wrapping an elastomeric casing around the open space between elastomer holding sleeve 1080 and bumper 1100 of cavity creation device 1000. The elastomeric casing is mechanically attached to both elastomer holding sleeve 1080 and bumper 1100 defining an enclosed cylindrical space therebetween.

The cavity is created by forcing biocompatible filler, such as prepared bone cement, from an approved mixing device through elastomer holding sleeve 1080 and into the elastomeric casing. The biocompatible filler may be forced against a bumper at an end of the elastomeric casing. The casing expands thereby pushing bone tissue outward to create a bone cavity completely filled by the elastomer-encased biocompatible filler implant. Once the elastomer-encased biocompatible filler implant has reached the desired size, the surgeon disengages elastomer holding sleeve 1080. The elastomer-encased biocompatible filler implant remains in the human body as an FDA approved implantable device.

2.0 Elastomers

Elastomeric materials (generally polymers) generally deform upon application of a deforming force and substantially resume their original dimensions after the deforming force is removed. In a preferred embodiment, the elastomeric materials may be elastomers. Elastomers are rubbery polymers that can be expanded upon application of a compressive force or stretched easily to high extensions and rapidly recover their original dimensions when the applied stress is released. Preferably, elastomers are semi-crystalline. For a general discussion of elastomers, see R. J. YOUNG & P. A. LOVELL, INTRODUCTION To POLYMERS 11 (2d ed., Nelson Thomes Ltd 2002), hereby incorporated herein by reference. In general, except in the case of thermoplastic elastomers, the polymeric backbone is crosslinked. Exemplary functional, physical, and mechanical characteristics of suitable elastomeric materials are described in more detail below.

2.1 Functional Characteristics of Elastomers

One of skill in the art can readily identify elastomers according to desired functional, physical, and mechanical characteristics. Elastomers are preferably biocompatible and have suitable expansion and elongation properties, glass transition temperatures, crystallinity, and crosslinking.

2.1.1 Biocompatibility

Preferably, elastomeric materials are compatible with mammalian tissue, suitable for implantation in mammals, interact with mammalian tissue in a nontoxic manner, and do not destroy the cellular constituents of the body fluid with which they interface. M. Szycher, et al., POLYURETHANE ELASTOMERS IN MEDICINE 234-244 (1990); S. Dumitriu & D. Dumitriu, BIOCOMPATIBILITY OF POLYMERS 100-158 (1990), both of which citations are hereby incorporated herein by reference.

Methods for testing biocompatibility are well known in the art, see e.g., the Food and Drug Administration's (FDA) guidance and International Organization for Standardization (ISO) 10993 standards, hereby incorporated herein by reference, which provides a set of criteria for testing biocompatibility of elastomers based on the duration of the implantation and the nature of the contact with the biomaterial. Other methods for testing biocompatibility are described in the literature. For cytotoxicity tests, see e.g., M. J. Menconi, et al., Molecular Approaches to the Characterization of Cell and Blood/Biomaterial Interactions, 7 J. CARDIAC SURG. 177-187 (1992); B. Sadd, et al., Interactions of Osteoblasts and Macrophages with Biodegradable and Highly Porous Polyesterurethane Foam and Its Degradation Products, 32 J. BIOMED. MATER. RES. 355-366 (1996); H. Oshima & M. Nakamura, A Study On Reference Standard for Cytotoxicity Assay of Biomaterials, 4 BIOMED. MATER. ENG. 327-332 (1992), each of which references is hereby incorporated herein by reference. The crystal violet, neutral red, or tryptan blue cell viability screening tool is a commonly used tests to determine toxicity, see e.g., S. A. Rosenbluth, et al., Tissue Culture Method for Screening Toxicity of Plastic Materials to be Used in Medical Practice, 54 J. PHARMACEUT. SCI. 156-159 (1965), hereby incorporated herein by reference. For examples of sensitization assays of irritation, see e.g., B. Huang, et al., Cellular Reaction to Vascugraft Polyesterurethane Vascular Prosthesis: In Vivo Study In Rats, 13 BIOMATERIALS 209-216 (1992); for examples of hemocompatibility and blood interaction assays, see e.g., AFNOR. Nor. Biological Evaluation of Medical Devices, Part 4: Selection of Tests for Interactions with Blood, AFNOR, Paris, 1994, hereby incorporated herein by reference.

Most preferably, the elastomers are manufactured using appropriate principles of current Good Manufacturing Practice (cGMP) regulations and meet the Food and Drug Administration's (FDA) guidance and International Organization for Standardization (ISO) 10993 standards or devices for implantation into tissue or bone for a limited duration of less than 24 hours.

2.1.2 Expansion/Elongation

Preferably, elastomers expand in volume upon application of a compressive force and elongate upon application of a stretching force. In general elastomer expansion is directly related to elastomer elongation.

Elastomer expansion and elongation can be measured according to the procedure set forth in American Society for Testing and Materials (“ASTM”) D412 “Standard Test Methods for Vulcanized Rubber and Thermoplastic Elastomers-Tension” or the corresponding Dow Corning Corporate Test Method (“CTM”) 0137 Å, both of which citations are hereby incorporated herein by reference. ASTM test procedures are compiled, reviewed, and published by the American Society for Testing and Materials, an independent and voluntary standards development organization.

Expansion of an elastomer is generally defined as the percentage of volume increase per unit of compressive pressure. Preferably elastomers exhibit an expansion percentage of from about 100 percent to about 1000 percent of their original volume upon application of a compressive pressure of from about 25 psi to about 500 psi to an elastomer of dimensions suitable for use in a cavity creation device.

Elongation is generally defined as the increase in extension produced by a tensile stress, expressed numerically as a fraction or percentage of the elastomer's initial length. Preferably elastomers exhibit an elongation percentage of from about 100 percent to about 1000 percent of their original length upon application of a retraction force of from about 25 psi to about 500 psi to an elastomer of dimensions suitable for use in a cavity creation device.

Preferably, upon removal of the stress that caused the expansion or elongation, elastomers used revert to dimensions of from about 100 percent to about 110 percent of their original volume or length, more preferably, of from about 100 percent to about 103 percent of their original volume or length.

2.1.3 Glass Transition Temperature

As an elastomer is cooled, it becomes more viscous and flows less readily. As the temperature is reduced further, the elastomer transitions from a rubbery consistency to a relatively hard polymer glass. The temperature range at which the elastomer undergoes the transformation from a rubbery consistency to a relatively hard polymer glass is known as the glass transition temperature. For a general discussion, see R. J. YOUNG & P. A. LOVELL, INTRODUCTION To POLYMERS 292-300 (2d ed., Nelson Thomes Ltd 2002), hereby incorporated herein by reference.

Preferably, the elastomer has a glass transition temperature higher than the temperature at which it will be used. ID. at 300. Thus, preferably, the glass transition temperature of the elastomer is lower than the body temperature of the mammal to be treated, preferably, below about 15° C., more preferably, below about 10° C.

The glass transition temperature of elastomers is determined by well-known procedures such as those described in R. J. YOUNG & P. A. LOVELL, INTRODUCTION To POLYMERS 292-298 (2d ed., Nelson Thornes Ltd 2002), hereby incorporated herein by reference. For example, the glass transition temperature can be determined by measuring the specific volume of a polymer sample as a function of the temperature. Kovacs, 30 J. POLY. SCI. 131 (1958), hereby incorporated herein by reference.

2.1.4 Crystallinity

In another preferred embodiment, elastomers are semi-crystalline (i.e., have a relatively low degree of crystallinity). R. J. YOUNG & P. A. LOVELL, INTRODUCTION To POLYMERS 300 (2d ed., Nelson Thornes Ltd 2002). The degree of crystallinity can be measured by well-known procedures, such as those described in ID. at 241-285, hereby incorporated herein by reference. The preferred degree of crystallization of elastomers will generally depend on the identity and desired properties of the elastomer. One of skill in the art might readily determine the degree of crystallization needed for a particular elastomer by measuring the physical properties of the elastomer as a function of crystallinity.

2.1.5 Crosslinking

In still another preferred embodiment, elastomers are lightly crosslinked. R. J. YOUNG & P. A. LOVELL, INTRODUCTION To POLYMERS 300 (2d ed., Nelson Thornes Ltd 2002). However, chemical crosslinks are less important when a thermoplastic is used as the elastomeric material because thermoplastic elastomers display advantageous elastomeric properties with little or no crosslinking. ID. at 303-306.

The degree of crosslinking can be determined by well-known methods, such as those described in ID. at 300-305 and references cited therein, hereby incorporated herein by reference. The preferred degree of crosslinking of elastomers will generally depend on the identity and desired properties of the elastomer. One of skill in the art might readily determine the degree of crosslinking needed for a particular elastomer by measuring the physical properties of the elastomer as a function of crosslinking.

2.2 Examples of Elastomers

Suitable elastomers include, but are not limited to, (1) general use elastomers, such as natural rubber, synthetic polyisoprene, styrene butadiene copolymers, and polybutadienes; (2) special elastomers, such as ethylene propylene and diene copolymers, isobutylene isoprene copolymers, haloisobutylene isoprene copolymers, nitrile butadiene copolymers, and polychloroprenes; (3) very special elastomers, such as high thermal and/or chemical resistance elastomers, such as polysiloxane or silicone elastomers, fluorocarbon elastomers (e.g., Viton® A, Viton B, Viton GF, and Viton GFLT, all commercially available from DuPont Dow Elastomers LLC), chloropolyethylene and chlorosulfonyl polyethylenes, polyacrylates, ethylene vinyl acetate elastomers, ethylene methyl acrylate, hydrogenated nitrile elastomers, and epichlorhydrin elastomers; and (4) thermoplastic elastomers, such as Surlyn® (made by DuPont), styrene-butadiene-styrene polymer (which comprises short block of polystyrene followed by a longer block of polybutadiene, followed by another short block of polystyrene), and segmented copolymers prepared by the reaction of diisocyanate with a prepolymer polyol and a short chain diol. An example of this preparation procedure is described in R. J. YOUNG & P. A. LOVELL, INTRODUCTION To POLYMERS 117-118 (2d ed., Nelson Thomes Ltd 2002), hereby incorporated herein by reference.

Useful chemical families of elastomers include, but are not limited to, silicon elastomers, polyurethane elastomers, saturated polyesters and co-polyesters, natural rubbers, polyvinyl chlorides, polyolefins, styrene copolymers, natural rubbers, hydrogels, polypeptide and collagen elastomers, polyphosphazenes, polyamides, polyacrylics, and fluorinated elastomers.

In one preferred embodiment, elastomers are high-consistency rubber silicone elastomers. Suitable high-consistency rubber silicone elastomers include, but are not limited to, crosslinked dimethyl and methyl-vinyl siloxane copolymers and Dow Corning Class VI Elastomers.

2.3 Sources of Elastomers

Elastomers may be readily available commercially. Elastomers also may be synthesized and characterized using well-known methods, for example see, R. J. YOUNG & P. A. LOVELL, INTRODUCTION To POLYMERS 15-240 (2d ed., Nelson Thomes Ltd 2002), hereby incorporated herein by reference. Commercial sources and methods to manufacture various elastomers are disclosed in D. J. CHAUVEL-LEBRET ET AL., Biocompatibility Of Elastomers, in POLYMERIC BIOMATERIALS 311-360 (Severian Dumitriu ed., 2nd ed. 2002), hereby incorporated herein by reference.

Dow Corning Class VI elastomers are commercially available from Dow Corning Corporation Corporate Center, PO Box 994, Midland, Mich. 48686-0994, United States.

2.4 Additives

Elastomers may contain various additives, for example, fillers, such as precipitated silica, chalks, and kaolins; opacifiers, such as barium sulfate; protective agents, such as amines and phenols; and crosslinking agents. D. J. CHAUVEL-LEBRET ET AL., Biocompatibility Of Elastomers, in POLYMERIC BIOMATERIALS 311-360 (Severian Dumitriu ed., 2nd ed. 2002), hereby incorporated herein by reference.

2.5 Sterilization of Elastomers

Preferably, before use, the elastomers will be sterilized. Sterilization techniques will vary depending on the identity of the elastomer and the preference of the surgeon or manufacturer. Methods for sterilization are well known in the art. For general discussions with references see, Barry Garfinkle & Martin Henley, Sterilization, in 2 REMINGTON: THE SCIENCE AND PRACTICE OF PHARMACY 1463-1486 (Alfonso R. Gennaro ed., 19th ed., 1995); D. J. CHAUVEL-LEBRET ET AL., Biocompatibility Of Elastomers, in POLYMERIC BIOMATERIALS 334-335 (Severian Dumitriu ed., 2nd ed. 2002), both of which citations are hereby incorporated herein by reference.

Suitable methods for sterilization of elastomers include, but are not limited to, exposure to ethylene oxide (500-540 mg/L) in 80 percent carbon dioxide at a relative humidity of over 35% for 3 hours at 35-41° C.; exposure to gamma radiation (e.g., 2.5 Mrad for 5 hours), see e.g., A. Pizzoferrato et al., In Vitro Biocompatibility of a Polyurethane Catheter After Deposition of Fluorinated Film, 16 BIOMATERIALS 361-367 (1995); B. Sadd, et al., Interactions of Osteoblasts and Macrophages with Biodegradable and Highly Porous Polyesterurethan Foam and Its Degradation Products, 32 J. BIOMED. MATER. RES. 355-366 (1996), both of which citations are hereby incorporated herein by reference; dry heat or autoclaving, see e.g., K. M. Miller & J. M. Anderson, In Vitro Stimulation of Fibroblast Activity by Factors Generated from Human Monocytes Activated by Biomedical Polymers, J. BIOMED. MATER. RES. 911-930 (1989); and use of antiseptic solutions, see e.g., M. J. Menconi, et al., Molecular Approaches to the Characterization of Cell and Blood/Biomaterial Interactions, 7 J. CARDIAC SURG. 177-187 (1992); J. A. Hunt et al., Effect of Biomaterial Surface Charge on the Inflammatory Response: Evaluation of Cellular Infiltration and TNF alpha Production, J. BIOMED. MATER. RES. 139-144 (1996), both of which citations are hereby incorporated herein by reference.

3.0 Introduction of a Biocompatible Filler into the Bone Cavity

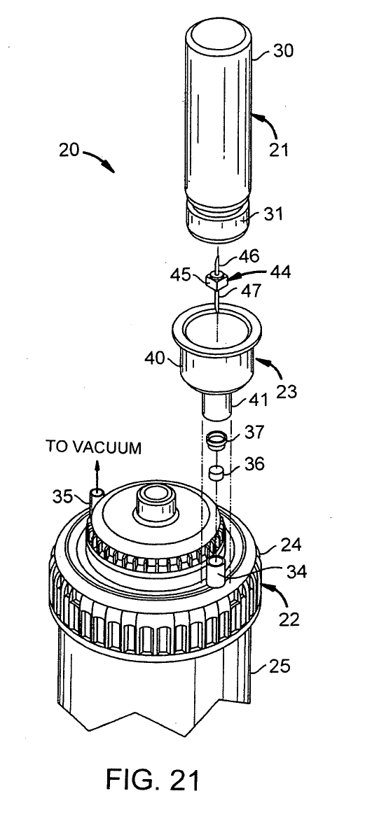

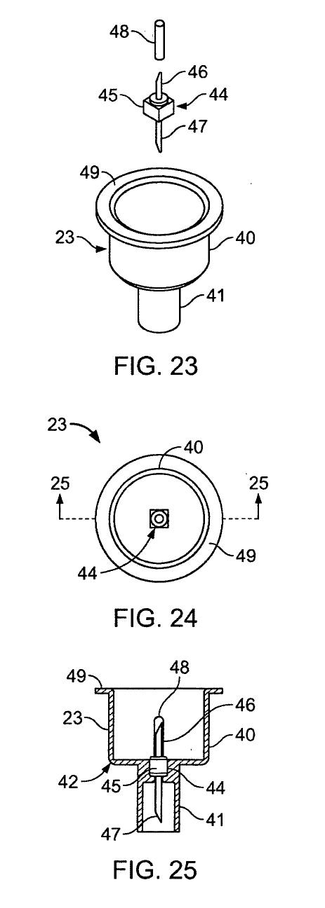

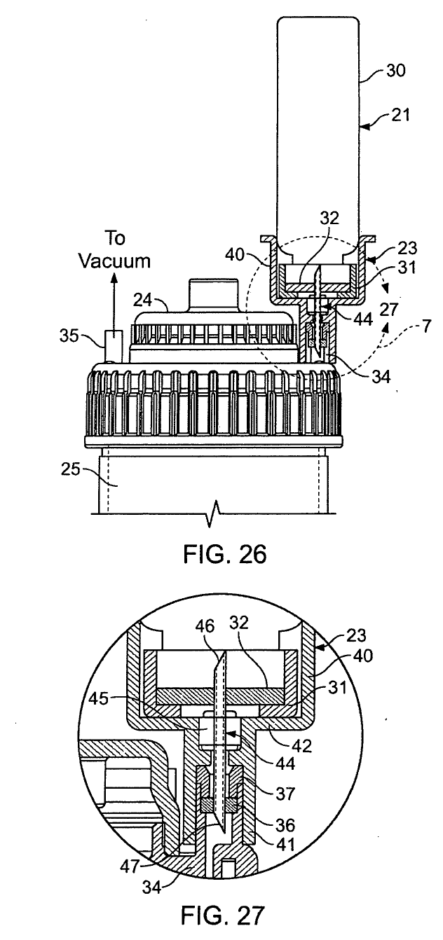

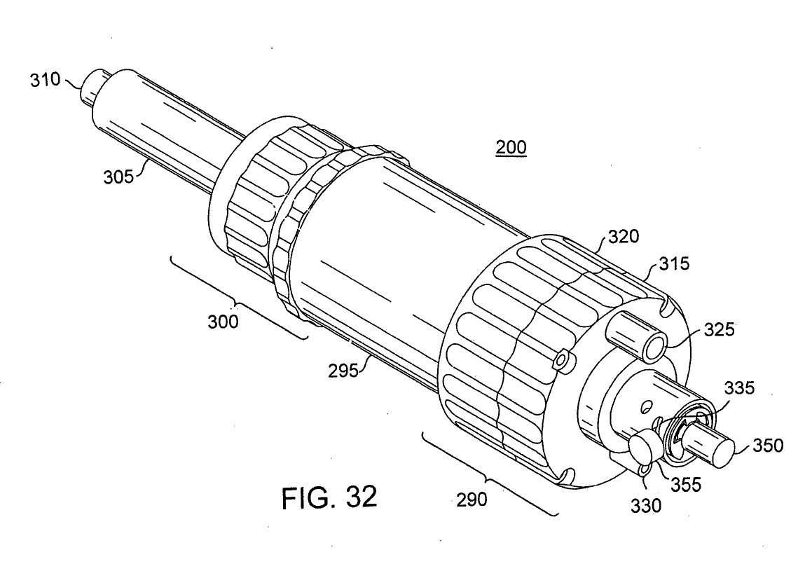

According to one embodiment, once the appropriate cavity is created in bone tissue, cavity creation device 1000 is removed from the delivery tube device 1050 and delivery tube device 1050 remains implanted in the bone tissue. A biocompatible filler, such as bone cement, can then be introduced into the cavity through the delivery tube device 1050 to treat weak, damaged, or diseased bone tissue. In one embodiment, a delivery device is connected directly to delivery tube device 1050 to deliver the biocompatible filler into the cavity. In a preferred embodiment, either the twistOR PrePack (illustrated by FIGS. 21-31) or Plexis™ (illustrated by FIGS. 32-40) systems described below are connected directly to delivery tube device 1050 and used to introduce a biocompatible filler.

3.1 twistOR PrePack™: An Exemplary Device for Introduction of Bone Cement Into a Bone Cavity

By referring to FIGS. 21-31, along with the following detailed discussion, the construction and operation of the preferred twistOR PrePack™ system to introduce a biocompatible filler, such as bone cement, into the bone cavity formed by the methods and devices discussed herein, can best be understood.

In FIGS. 21, 22, 26, and 27, twistOR PrePack™ system 20 is fully depicted as comprising container 21, integrated bone cement handling and delivery system 22, and transfer assembly 23, preferably, a fluid transfer assembly. Container 21 is preferably a sealed container, more preferably, a sealed container designed for containing corrosive chemicals, such as liquid monomer. As used herein, “sealed” means that the container's contents are prevented from leaking during handling and transport and are protected from air. As shown, integrated bone cement handling and delivery system 22 comprises cover 24 that is threadedly mounted to vessel 25.

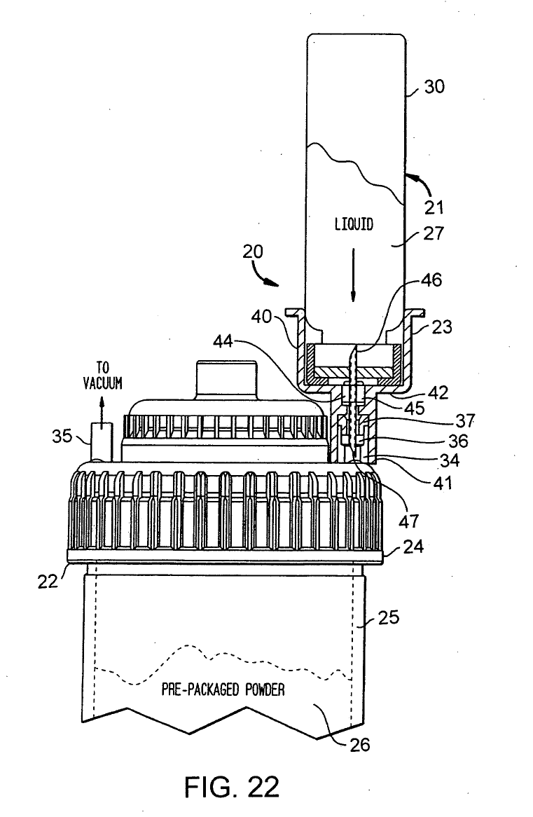

In the preferred construction, the second component of the bone cement, which comprises dry powder 26, is stored in vessel 25 of bone cement handling and delivery system 22, as clearly shown in FIG. 22. However, if desired, dry powder 26 may be stored in any suitable container, bag, or pouch that is opened just prior to use with the powder being added to vessel 25.

In addition to preferably shipping dry powder 26 in vessel 25 of bone cement handling and delivery system 22, the first component, which comprises liquid monomer 27, is contained in sealed container 21. Sealed container 21 can be any suitable container adaptable to create a flow path to the vessel by way of transfer assembly 23. For example, sealed container 21 can be flexible or non-flexible plastic or polymer, preferably, glass or other chemically resistant material. In one preferred embodiment, sealed container 21 comprises glass vial or tube 30 having a single opening or portal on which cap or closure 31 is mounted.

As detailed above, cap or closure 31 of sealed container 21 comprises an integrally formed sealing membrane, preferably, a septum to provide access to the interior of glass vial/tube 30. Sealing membrane 32 comprises a generally conventional construction, formed of elastomeric material, which typically comprises elastomeric plastics, rubbers, silicones, and the like. In this way, liquid monomer 27 is sealed within glass tube/vial 30, while providing access to the interior of tube/vial 30 only upon creating a flow path, for example, by using a transfer conduit, such as a suitable syringe needle.

In certain embodiments, vacuum is used to cause the sealed-container contents to transfer into the vessel (the means for transfer). In these embodiments, the vessel will comprise vacuum portal 35 for attachment to a vacuum supply. In other embodiments, sealed container 21 can be constructed such that the twistOR PrePack™ can operate without vacuum. Sealed container 21 will comprise the means to transfer the container contents into vessel 25. In these embodiments, vacuum portal 35 is not required. In one such embodiment, sealed container 21 is a chemically resistant squeeze bottle or flexible bag so that container 21's contents can be squeezed into the vessel 25. In another such embodiment, sealed container 21 is preloaded with a pressurized gas that functions to push the monomer out of container 21 upon creating a flow path by connection to transfer assembly 23. Preferably, container 21's contents (e.g., monomer) is preloaded along with the pressurized gas.

In addition, cover 24 of bone cement handling and delivery system 22 comprises an access portal 34 and vacuum portal 35 that are mounted thereto and provide access to the interior of vessel 24. Vacuum portal 35 comprises a generally conventional construction that enables a vacuum source to be connected thereto, using any suitable vacuum connection. In addition, access portal 34 comprises a sealing membrane 36, preferably, a septa-like disk mounted in access portal 34 for sealing the interior of vessel 25 from the ambient air, while also enabling access to the interior of vessel 25 to be achieved by creating a flow path, for example by employing a transfer conduit, such as a suitable needle or syringe.

Finally, holder 37 is employed for maintaining sealing membrane 36 in the precisely desired position within access portal 34. By forming holder 37 with two separate and distinct diameters, one portion of holder 37 is inserted into access portal 34, while the second, larger diameter portion thereof engages the outer terminating edge of access portal 34. In this way, sealing membrane 36 is securely maintained in the desired position within access portal 34.

The construction of transfer assembly 23 of twistOR PrePack™ is completed by providing for mating engagement thereof with cap 31 of sealed container 21 and access portal 34 of cover 24 of handling and delivery system 22. As fully depicted in FIGS. 21-27, in its preferred embodiment, transfer assembly 23 comprises collar portions 40 and 41, interconnected with each other along support plate 42. In addition, collar portions 40 and 41 preferably comprise generally cylindrical shapes and are coaxially aligned with each other.

In addition, collar portion 40 is constructed with an inside diameter dimensioned for co-operative, frictional engagement with cap 31 of sealed container 21. In this way, when transfer assembly 23 is mounted to sealed container 21, transfer assembly 23 is frictionally engaged securely with sealed container 21, preventing any unwanted, easy dislodgment of sealed container 21 from assembly 23.

Similarly, collar 41 comprises an inside dimension constructed for mating, co-operative, sliding engagement with access portal 34 of cover 24. In addition, by designing collar 41 with an inside dimension that is slightly greater than the outside dimension of access portal 34, secure holding engagement of transfer assembly 23 with access portal 34 is achieved whenever assembly 23 is telescopically mounted into overlying engagement with access portal 34.

In order to complete the construction of transfer assembly 23, a mechanism for providing a flow path between the vessel and the sealed container, is provided. The preferred flow path is created by a transfer conduit, such as dual ended piercing conduit 44 (double-tipped syringe needle). As depicted, transfer conduit 44 comprises a support base 45, a syringe needle forming member 46 mounted to one surface of support base 45 and a syringe needle forming member 47 mounted to the opposed surface of support base 45.

In the preferred construction, syringe needle forming members 46 and 47 comprise elongated, hollow tubes mounted to support base 45 in coaxial alignment with each other, forming a continuous, elongated flow path therebetween. In addition, each syringe needle forming member 46 and 47 comprises sharp, pointed, distal ends constructed for piercing the sealing membrane 36 (any septa-like material) for gaining access to the interior associated with the sealing membrane.

In addition, base 45 of piercing element 44 is securely mounted in transfer assembly 23, preferably affixed in support plate 42. When mounted in its secure position, syringe needle forming member 46 extends into collar portion 40, substantially centrally disposed therein. In this position, syringe needle forming member 46 is peripherally surrounded by the wall forming collar portion 40 with its sharp, distal end extending toward the opening of collar 40.

Similarly, syringe needle forming member 47 is securely positioned to be centrally disposed within collar portion 40, peripherally surrounded by the wall forming collar 41. In addition, the sharp distal end of syringe needle forming portion 47 extends towards the open end of collar 41.

By employing this construction, the telescopic axial advance of transfer assembly 23 into engagement with sealed container 21 and access portal 34 of cover 24, causes syringe needle forming portions 46 and 47 to pierce the sealing membranes 32 and 36 and establish a direct fluid transfer flow path between sealed container 21 and vessel 25. In the preferred construction, in order to eliminate any unwanted injuries, tip cover 48 is preferably mounted to syringe needle forming member 46. Since the diameter of collar portion 40 is large enough to enable a finger tip to enter its open end, the use of cover 48 prior to engagement of cover 40 onto cap 31 provides the desired protection.

In addition, in the preferred construction, collar 40 comprises radially extending flange 49 formed on its terminating end. By employing flange 49, ease of use and control of collar 40 is provided.

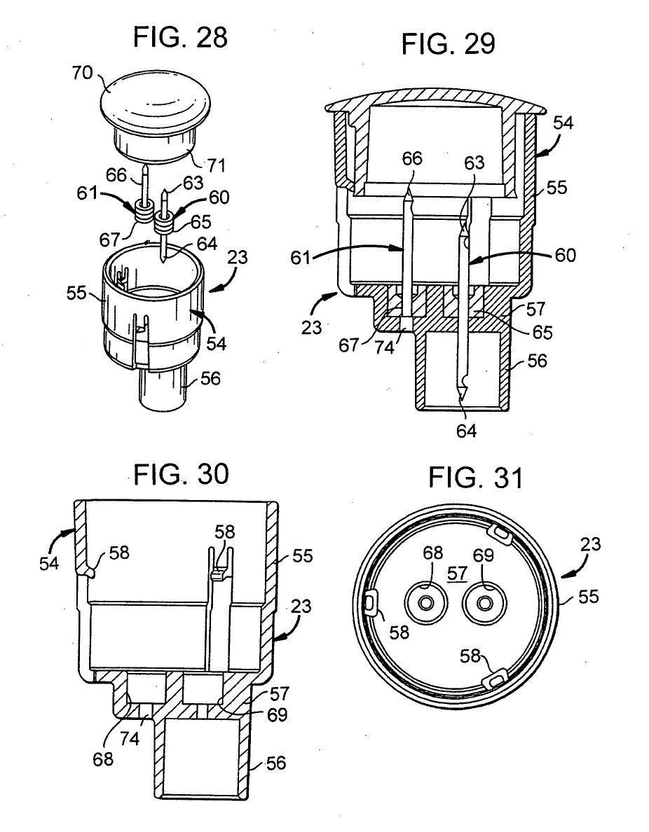

By referring to FIGS. 28-31, along with the following detailed discussion, the construction of an alternate, preferred embodiment of twistOR PrePack 23 is provided. In this embodiment, transfer assembly 23 comprises a housing 54 that incorporates collar portions 55 and 56, interconnected to each other by support wall 57. In the preferred embodiment, collar portions 55 and 56 preferably comprise generally cylindrical shapes and are vertically aligned with each other. In addition, the central axis of each collar portion is parallel to each other and offset from each other.

As with the embodiment detailed above, collar portion 56 comprises an inside diameter constructed for mating, co-operative, sliding engagement with access portal 34 of cover 24. In addition, by designing collar portion 56 with an inside diameter that is slightly greater than the outside diameter of access portal 34, secure holding engagement of transfer assembly 23 with access portal 34 is achieved whenever assembly 23 is telescopically mounted into overlying engagement with access portal 34.

In addition, collar portion 55 comprises an inside diameter dimensioned for co-operative, frictional engagement with cap 31 of sealed container 21. In addition, in this embodiment, collar portion 55 comprises a plurality of tabs 58 mounted to the inside wall of collar portion 55 that extend radially inwardly therefrom. In addition, tabs 58 are formed on the inside wall of collar portion 55 in a vertical position that is slightly greater than the vertical height of cap 31 of sealed container 21. Finally, in the preferred construction, tabs 58 are formed about the inside wall of collar portion 55 substantially equidistant from each other, thereby being spaced apart a distance of about 120°.

By employing this construction, whenever sealed container 21 is telescopically inserted into collar portion 55 of transfer assembly 23, cap 31 of sealed container 21 is frictionally engaged with collar portion 55, securely locked in position by tabs 58 engaging the edge of cap 31 and preventing telescopic removal of sealed container 21 from collar portion 55. In this way, once sealed container 21 has been mounted in secure, locked engagement with transfer assembly 23, dislodgment or removal of sealed container 21 from collar 55 is prevented.

Furthermore, in this embodiment of twistOR PrePack™, transfer assembly 23 comprises gas-flow aperture 74 comprising gas-flow conduit 61 mounted in support wall 57 and transfer conduit 60 also mounted in support wall 57. Preferably, transfer conduit 60 and gas-flow conduit 61 are independent syringe needles. As shown in FIGS. 28 and 29, transfer conduit 60 comprises an elongated, continuous, tubular member that defines an elongated flow path and incorporates two separate and independent piercing ends 63 and 64 mounted to support base 65. In another embodiment, conduit 60 is molded directly into housing 54 and, thus, support base 65 is not required.

With support base 65 of transfer conduit 60 mounted in receiving hole 69 of support wall 57 of transfer assembly 23, piercing end 63 extends from support wall 57 into the interior of collar portion 55, while piercing end 64 extends from support wall 57 into collar portion 56. In this way, as detailed above, whenever transfer assembly 23 is mounted to access portal 34 of cover 24, and sealed container 21 is mounted to transfer assembly 23, the monomer contained in sealed container 21 is able to be transferred through transfer conduit 60 into vessel 25.

In this embodiment of twistOR PrePack™, transfer assembly 23 also comprises a gas-flow conduit 61 that incorporates an elongated, cylindrically shaped, hollow piercing element 66 mounted to support base 67. In the preferred construction, support base 67 is mounted in receiving hole 68 formed in support wall 57 of transfer assembly 23, with hollow piercing element 66 extending therefrom into the interior of collar portion 55. In addition, base 67 of gas-flow conduit 61 cooperates with gas-flow aperture 74 formed in support wall 57, thereby providing an air flow path from the ambient surroundings through hollow gas-flow conduit 61 into the interior of sealed container 21 whenever sealed container 21 is mounted in collar 55.

By employing this embodiment of twistOR PrePack™ 23, it is ensured that monomer stored in sealed container 21 is capable of flowing freely through transfer conduit 60 into vessel 25 whenever the monomer is desired for being added into vessel 25. By providing a separate gas flow pathway (preferably ambient air) through gas-flow aperture 74 and gas-flow conduit 61, gas, such as nitrogen, argon, or other inert gas or air is constantly replaced in sealed container 21 as the monomer is withdrawn therefrom. In this way, the creation of a partial vacuum is avoided and free flow of the monomer is provided.

In the preferred construction, this embodiment of twistOR PrePack™ is completed by incorporating cover 70 that is constructed for being mounted in collar portion 55 for preventing and blocking any unwanted entry into collar portion 55, prior to the insertion of sealed container 21. In this way, contact with the terminating ends of piercing elements 63 and 66 is prevented and any unwanted or accidental injury is avoided.

In the preferred construction, cover 70 comprises an outwardly extending rim 71 formed on the base thereof, which cooperates with inwardly extending tabs 58, in order to secure cover 70 in the desired position. In addition, whenever monomer bearing sealed container 21 is ready for insertion in collar portion 55, cover 70 is easily removed from its secured position, thereby enabling sealed container 21 to be telescopically inserted and locked in position in collar portion 55.

3.2 The Plexis™ System: An Exemplary Device for Introduction of Bone Cement into a Bone Cavity

FIGS. 32-38 and the corresponding text below provide a detailed disclosure of the construction and operation of a Plexis™ system to introduce a biocompatible filler, such as bone cement, into the bone cavity formed by the methods and devices discussed herein.

In operation, Plexis™ system 200 corresponds to bone cement handling and delivery system 22 of FIGS. 21-31 and as discussed in detail above. Transfer of liquid monomer under vacuum to Plexis™ system 200 is substantially similar to the transfer procedure described above for vessel 25. Thus, the Plexis™ system is preferably used in conjunction with sealed container 21 and fluid transfer assembly 23, (both of FIGS. 21, 22, 26, and 27).

FIG. 32 depicts one embodiment of a fully assembled Plexis™ system 200. Apparatus 200 comprises mixing chamber 295, controllable portal assembly 300, and dispensing chamber 305, preferably, tube shaped, having dispensing portal 310. Preferably, dispensing portal 310 is adapted to connect to the standard needle or cannula used in vertebroplasty procedures. Controllable portal assembly 300 comprises a controllable portal discussed in more detail below, which provides controlled opening of a flow path between the sealed mixing chamber 295 and dispensing chamber 305. In a preferred embodiment, mixing chamber 295 comprises cover assembly 290. Preferably, cover assembly 290 comprises top cap 315 attached to mixing-chamber cover 320 by way of set screws. Mixing chamber 295 comprises access portal 325, vacuum portal 330, and preferably comprises engagement-pinslot 335 for receiving engagement pin 355.

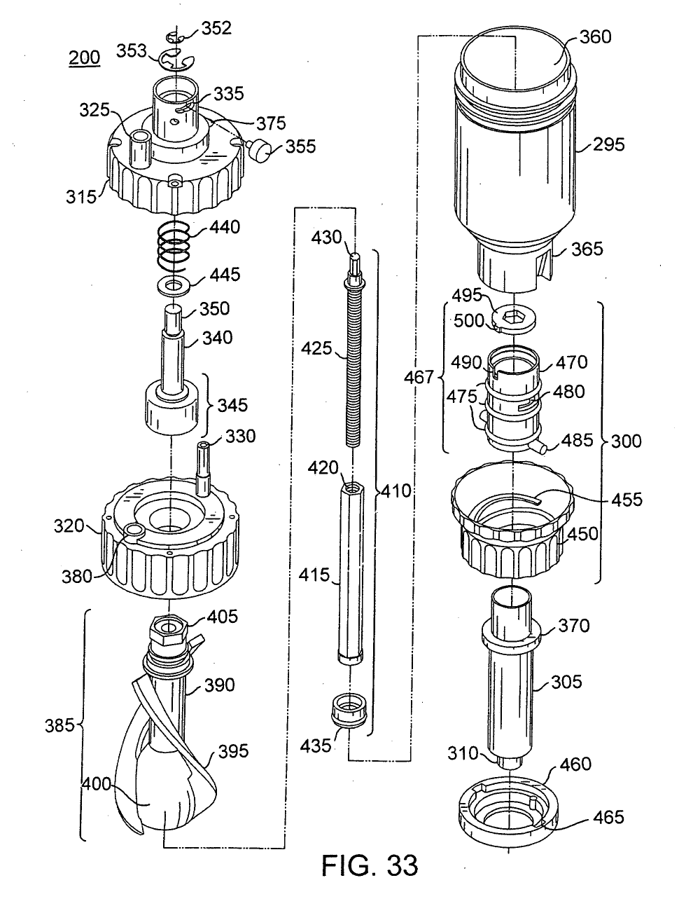

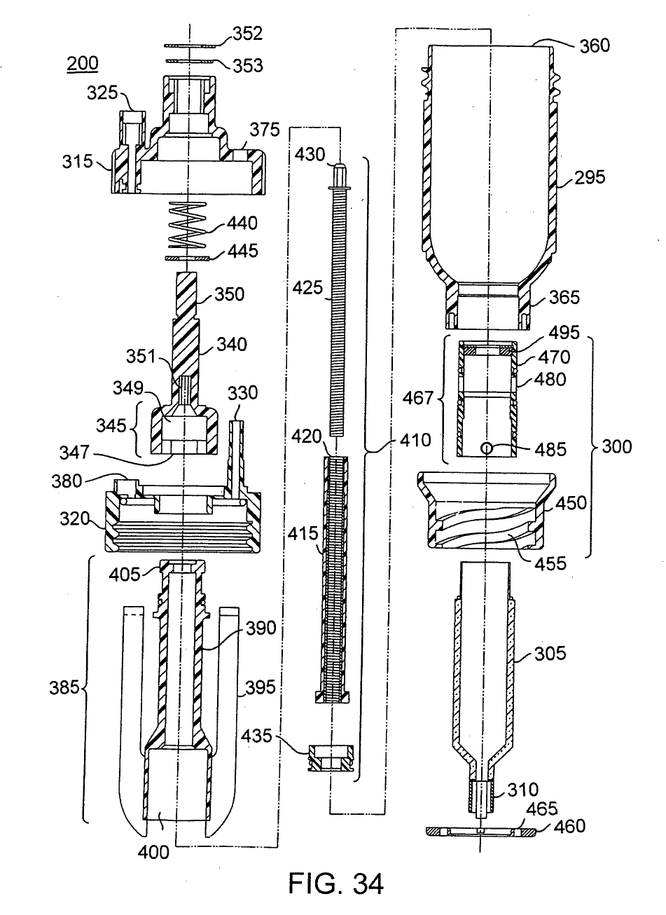

FIGS. 33 and 34 are exploded and cross-section side elevation views of apparatus 200 depicting the interrelation of component parts in a preferred embodiment of the Plexis™ system. As illustrated in FIG. 33, mixing chamber 295 defines mixing cavity 360 for receiving the separate components to be mixed and dispensed. Preferably, mixing chamber 295 comprises a smaller-diameter end 365 to receive controllable portal assembly 300. Dispensing chamber 305 is connected to mixing chamber 295. When the controllable portal housed in controllable portal assembly 300 is closed, sealed mixing chamber 295 is isolated from dispensing chamber 305. On the other hand, opening the controllable portal creates a flow path so that dispensing chamber 305 can receive mixed components from mixing chamber 295 for dispensation. Preferably, dispensing chamber 305 comprises support flange 370.

As discussed above, in a preferred construction, mixing chamber 295 comprises cover assembly 290 (see FIG. 32), which, in turn, comprises end cap 315 and a mixing-chamber cover 320. In this embodiment, as shown in FIG. 33, end cap 315 comprises opening 375 aligned with vacuum portal 330, and mixing-chamber cover 320 comprises opening 380 aligned with access portal 325.

In a preferred embodiment of cover assembly 290, mixing chamber cover 320 attaches to mixing chamber 295 by threaded engagement. Mixing chamber 295 houses mixing-unit 385. Mixing unit 385 can be any assembly well known in the art to mix components, for example, but not limited to, mixers comprising mixing vanes, such as paddles, blades, and propellers. Preferably, mixing unit 385 comprises cylindrical, hollow mixing shaft 390 and helical mixing vanes 395. In a more preferred embodiment, hollow mixing shaft 390 comprises a large-diameter end 400 and mixing head 405.

The Plexis™ system further comprises a drive mechanism to drive the mixed components from dispensing chamber 305 into the desired location. The drive mechanism can be any device well known in the art to drive contents from a chamber. Preferably, the drive mechanism comprises a plunger that can be driven by a rotational drive or simply by pushing the plunger down by hand.

The preferred drive mechanism 410 is shown in FIG. 33, which comprises plunger shaft 415 having bore 420, which houses axially-movable plunger shaft advancing member 425. Preferably, plunger advancing member 425 terminates in drive head 430 constructed for rotational engagement with drive-head engagement 351. Preferably, advancing-member 425 comprises male threads, and bore 420 comprises complimentary female threads. Preferably, plunger shaft 415 comprises plunger-sealing-end 435. Preferably, plunger-sealing-end 435 is constructed of a flexible, chemically resistant material and has a diameter slightly greater than the inner diameter of dispensing chamber 305 to ensure that all of the material contained within dispensing chamber 305 is axially advanced upon movement of plunger shaft 415. Preferably, drive mechanism 410 is housed by hollow mixing shaft 390.

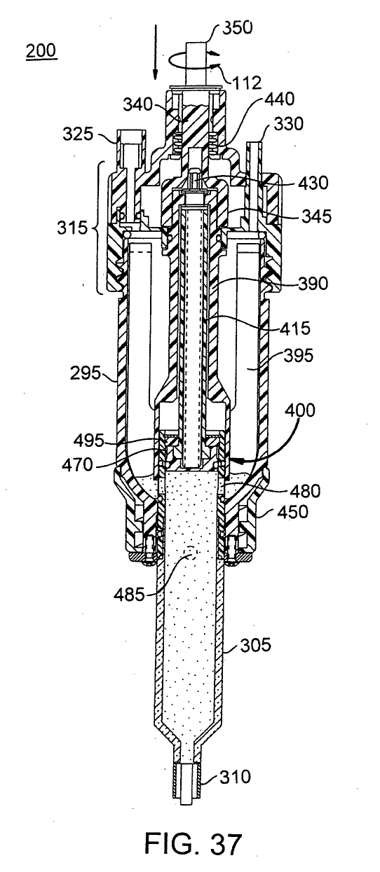

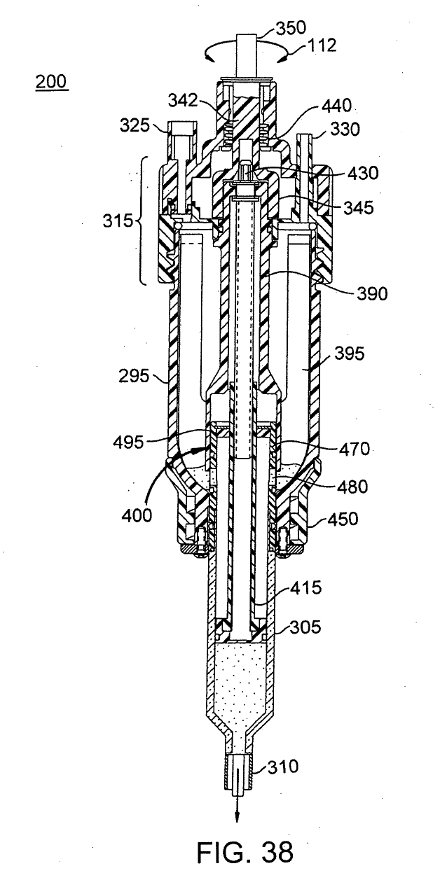

Rotational drive 112 (shown in FIGS. 37-38 as an arrow indicating rotational movement) connects to rotating-means connection 350 of drop shaft 340. Rotating-means connection 350 is firmly secured to end cap 315 by lock washers 352 and 353. Rotational drive 112 can be any motorized or manually driven rotating device inducing rotation, which are well known in the art, for example, but not limited to a drill, handle, or hand crank. In the mixing stage, rotational drive 112 rotates mixing unit 385 by way of drop shaft 340. This is because, in the mixing stage, the lower portion 347 (see FIG. 34) of mixing unit connection 345 is engaged with mixing head 405. Mixing unit connection 345 comprises a lower portion 347 (see FIG. 34) having an interior configuration that is geometrically complementary to mixing head 405 (e.g., hexagonal) so as to rotationally engage the mixing head 405 (e.g., a hexagonal shape) and an upper portion 349 (see FIG. 34) having an interior configuration that will not engage mixing head 405 (e.g., a smooth round shape). Mixing unit connection 345 is designed in this manner so that when drop-shaft 340 is in the up position (mixing phase), mixing head 405 and drop-shaft 340 are rotationally engaged by way of complementary geometries between the lower portion 347 of mixing unit connection 345 and mixing head 405. On the other hand, after mixing is complete and the mixing chamber contents have been transferred to dispensing chamber 305, drop-shaft 340 is dropped, whereby the smooth round upper portion 349 (FIG. 34) of mixing unit connection 345 is adjacent to mixing head 405 and, in effect, drop-shaft 340 is disengaged from mixing head 405. Thus, rotation of drop-shaft 340 does not rotate mixing unit 385. This dispensing phase is explained in more detail below.

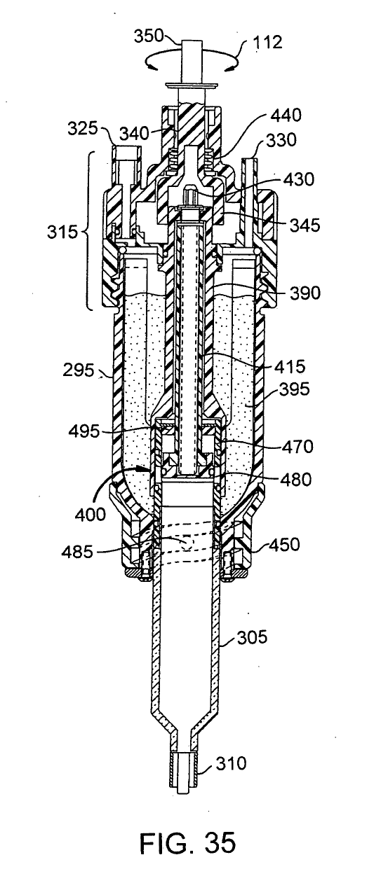

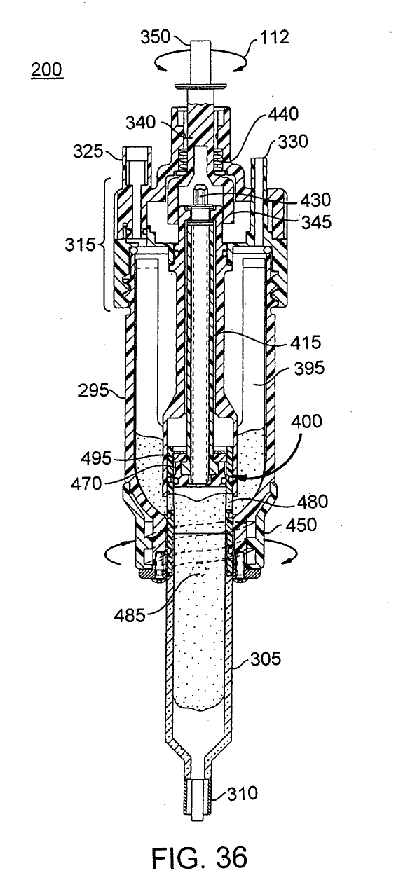

During the mixing stage, drop shaft 340 is in the up position such that drive-head engagement 351 is held above and is therefore not engaged with drive head 430. This is illustrated by FIGS. 35 and 36. At the point when dispensation is desired, however, by a simple mechanical adjustment (i.e., disengaging engagement pin 355), drop shaft 340 is forced down by the action of spring 440 and washer 445 with the result that the lower portion 347 (FIG. 34) of mixing unit connection 345 disengages from mixing head 405 and, at the same time, drive-head engagement 351 of drop shaft 340 engages with drive mechanism 410 by way of drive head 430. Then activation of rotational drive 112 controllably advances plunger 415. This aspect of the embodiment is illustrated by FIGS. 37 and 38.

As mentioned above, controllable portal assembly 300 comprises a mechanism for opening a flow path between mixing chamber 295 and dispensing chamber 305 after mixing of the components contained in mixing chamber 295 is complete. Such a mechanism is herein termed a controllable portal. FIG. 33 depicts a preferred controllable portal assembly 300 comprising locking collar 450, having threads 455, and end cap 460 having locking slots 465. Controllable portal assembly 300 connects to the base of mixing chamber 295. The controllable portal can be any valve, stopcock, or other device effective to isolate the contents of mixing chamber 295 from dispensing chamber 305 during the mixing phase and also to create a flow path between mixing chamber 295 and dispensing chamber 305 when transfer between mixing chamber 295 and dispensing chamber 305 is desired. A preferred embodiment of a controllable portal is depicted in FIG. 33 as 467.

Controllable portal 467 comprises sliding tube 470 securely fixed to dispensing chamber 305. Preferably, sliding tube 470 forms a tight seal with both the mixing chamber 295 and dispensing chamber 305, for example, by use of o-rings 475. In FIG. 33, sliding tube 470 comprises a pair of windows 480 on each side and radially extending locking rods 485. Sliding tube 470 further comprises plunger-locking-slot 490. Sliding tube 470 can be an integral part of dispensing chamber 305 or can be a separate component for secure, fixed attachment to dispensing chamber 305. In a preferred embodiment, radially extending locking rods 485 are positioned for cooperating, controlled, sliding engagement with threads 455 of locking collar 450. Guide washer 495 is designed to be geometrically complementary to plunger shaft 415 so as allow plunger shaft 415 to move up and down along its axis but not to rotate. Guide washer 495 comprises tooth 500 complementary in shape to plungerlocking-slot 490.

3.2.1 The Mixing Phase of the Plexis™ System

The components to be mixed are contained within mixing chamber 295. One or more of the components can be prepackaged in the mixing and dispensing unit and/or additional components can be added directly before mixing.

As shown in FIG. 35, during the mixing phase, sliding tube 470 is positioned by threads 455 of locking collar 450 so that: (1) windows 480 are within large-diameter end 400 of hollow mixing shaft 390; and (2) the flow path (i.e., windows 480) between mixing chamber 295 and dispensing chamber 305 is blocked. In other words, the interior of dispensing chamber 305 is isolated from the interior of mixing chamber 295, preventing the contents from entering dispensing chamber 305 during mixing.

Further, in this mixing phase, drop shaft 340 is engaged by engagement pin 355 and therefore locked in the up position such that drive head 430 is not engaged with rotating drive-head engagement 351. And in the up position, as discussed above, drop shaft 340 is rotationally engaged with mixing head 405. Also, advancing member 425 is fully inserted into bore 420. Tooth 500 of guide washer 495 is engaged with locking-slot 490 so that plunger shaft 415 is prevented from rotating.

In the above configuration, upon connection and operation of a rotational drive 112 to rotating-means connection 350, mixing unit 385 is rotated along its axis thereby mixing the components within mixing chamber 295.

3.2.2 Transfer of Mixed Components from Mixing Chamber to Dispensing Chamber Of the Plexis™ System

When the mixing phase is complete, the contents of mixing chamber 295 are ready for transfer to dispensing chamber 305. This is accomplished by opening controllable portal 467 to create a flow path. In a preferred embodiment, rotation of helical shaped mixing vanes 395 is used force the contents of mixing chamber 295 into dispensing chamber 305 by action of mixing unit 385.

FIGS. 35 and 36 illustrate operation of controllable portal 467 to open a flow path between mixing chamber 295 and dispensing chamber 305 and using the action of mixing unit 385 to transfer the contents. First locking collar 450 is rotated whereupon locking rods 485 are guided within threads 455 of locking collar 450 thereby pushing sliding tube 470 and dispensing chamber 305 downward such that windows 480 are below plunger-sealing-end 435 and a flow path between mixing chamber 295 and dispensing chamber 305 is created. Thus, the axial rotational movement of locking collar 450 causes windows 480 of sliding tube 470 to move out of engagement with the larger diameter end 400 of hollow mixing shaft 390, whereby windows 480 are positioned below plunger-sealing-end 435 to complete the flow path.

Rotating of locking collar 450 is complete when locking rods 485 are locked within complementary locking slots 465 of end cap 460. The construction of locking rods 485 and locking collar 450 effectively provide a turnbuckle construction that causes dispensing chamber 305 to move downward.

Once dispensing chamber 305 is in the position depicted in FIG. 36, rotational drive 112 is activated to force the contents of mixing chamber 295 into dispensing chamber 305 by 30 the helical action of mixing unit 385.

3.2.3 The Dispensing Phase of the Plexis™ System

Once the contents are loaded into dispensing chamber 305, drop shaft 340 can be dropped by releasing engagement pin 355. This causes drive-head engagement 351 of drop shaft 340 to rotationally engage with drive head 430 of plunger advancing member 425. At the same time the upper portion 349 (FIG. 34) of mixing unit connection 345, having a smooth interior (not shown), drops over mixing head 405 and the geometrically complementary lower portion 347 (FIG. 34)of connection 345 disengages from mixing head 405. Accordingly, in this position, the rotation of drop-shaft 340 does not rotate mixing unit 385. Dispensing the contents of dispensing chamber 305 is illustrated in FIGS. 37 and 38.

Upon activating rotational drive 112, rotating means connection 350 is controllably rotated. The rotational movement causes plunger advancing member 425 to rotate. Since plunger advancing member 425 is axially fixed (cannot move up and down but can only rotate), plunger shaft 415 and plunger-sealing-end 435 are controllably axially advanced longitudinally through dispensing chamber 305. The longitudinal movement of plunger sealing-end 435 in dispensing chamber 305 forces the mixed components contained therein to be delivered through outlet portal 310 of dispensing chamber 305. Preferably, dispensing portal 310 is adapted to connect to the standard needle or cannula (not shown) used in vertebroplasty procedures.

In addition, by controlling the rotational movement or speed of rotating-means connection 350, the precisely desired pressure for advancing the mixed components through dispensing chamber 305 is achieved. Furthermore, by stopping the rotational movement of rotating-means connection 350 or reversing the direction rotating-means connection 350, complete control over the delivery of the mixed components to the precisely desired site is achieved. In fact, by reversing the rotation of rotating-means connection 350, the plunger direction is reversed and the contents can actually be pulled back into dispensing chamber 305. This provides much greater control than previously available. In addition, in the preferred embodiment, reference indicia are marked or etched on the outer surface of dispensing chamber 305, thereby enabling the operator to precisely measure the quantity of material being delivered.

In another convenient embodiment, the Plexis™ system can be calibrated such that the number of revolutions of drop shaft 340 and/or the rotational drive 112 corresponds to an amount (e.g., a weight or volume) of bone cement dispensed. In this embodiment, a clinician dispensing a biocompatible filler using the Plexis™ system can dispense a predetermined amount by completing a pre-determined number of rotations of drop shaft 340 and/or rotational drive 112.

3.3 Illustrative Embodiment—Manually Actuable Plexis™ System

Certain aspects may now be more clearly understood by consideration of the following specific embodiment in the form of a manually actuable Plexis™ system. By manually actuable is meant that both mixing of the components and dispensing of the mixed components can be manually effected and controlled without the use of power tools. One advantage of manual actuation is that operation is not dependent on the presence of power tools or electrical outlets in a sterile environment. Another is the finer level of control provided by direct hand control.

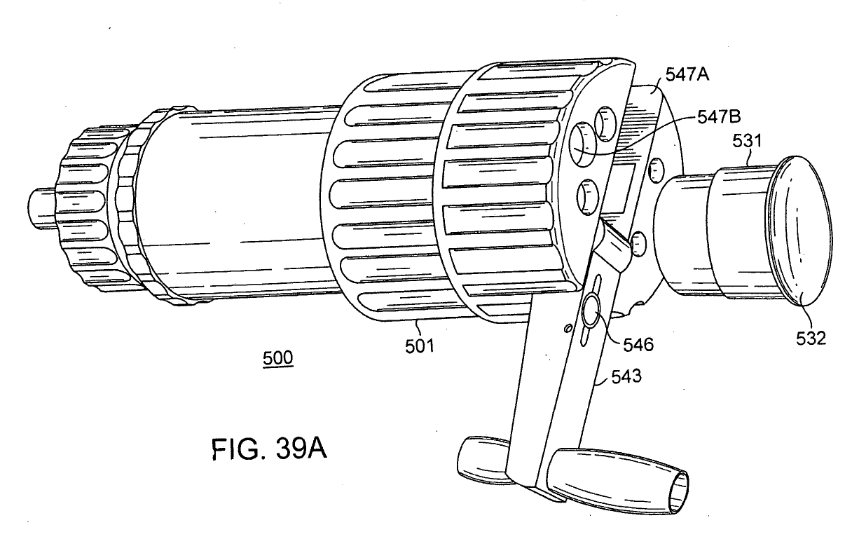

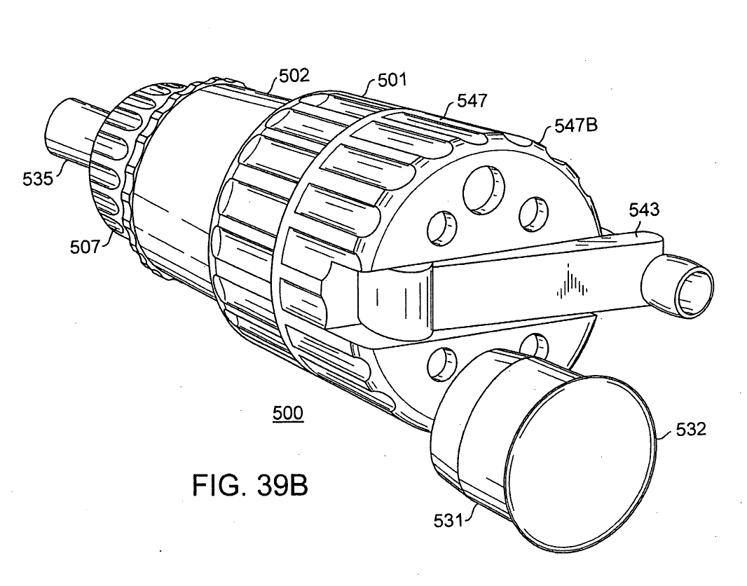

The exemplary apparatus is similar in structure and function to the apparatus previously illustrated and described except that it provides adaptations to facilitate manual mixing and manual dispensing of the mixed components. FIG. 39A is a perspective view of the assembled exemplary apparatus 500 with a radially extending handle 543 in a first position to facilitate manual mixing of liquid monomer and bone cement powder. The handle 543 is pivotally mounted on a handle cap 547. FIG. 39B shows the same apparatus 500 with the handle 543 pivoted to a second position to facilitate dispensing the mixture. The assembled apparatus 500 comprises a mixing chamber 502, controllable portal assembly 507, and dispensing chamber 535, preferably tube shaped, having a dispensing portal (not visible). Preferably the dispensing portal is adapted to connect to the standard needle or cannula used in vertebroplasty procedures. The controllable portal assembly 507 provides controlled opening of a flow path between the sealed mixing chamber 502 and dispensing chamber 535. Preferably, mixing chamber 502 comprises a top cap 501.

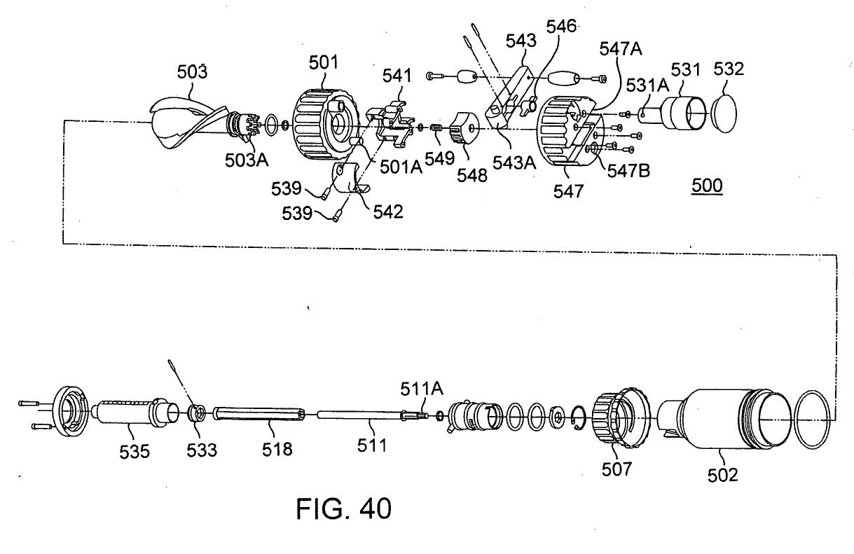

FIG. 40 is an exploded view of a manually actuable Plexis™ system 500. The apparatus is similar in operation and structure to those already described herein. The discussion here will emphasize the modifications found advantageous for manual actuation.

It is contemplated that the apparatus 500 will be delivered with the crank handle 543 in the open radially extended position (FIG. 39A) and the needle transfer housing 531, 532 in place. The user will remove the cover 532 of the needle transfer housing 531 and attach a vacuum source (not shown) to the vacuum port 501A as previously described. With vacuum applied, needles of the transfer housing pierce the cap of a monomer vial and liquid monomer from the vial is drawn into the transfer housing 531.

The monomer is further drawn into the mixing housing 502 which can contain the powder polymer. The vial and the transfer housing 531 are then removed together, the simultaneous removal assured by locking fingers of the transfer housing locking onto the cap of the vial.

The user then mixes the monomer and powder to form bone cement by rotating the radially extended handle 543 or by rotating the handle cap 547 (which acts as a knob). With the handle 543 in open position, a cam surface 543A pushes downward on a crank handle gear 548 causing the gear configuration to interface with a corresponding gear configuration 503A on the end of mixing paddle 503. The manual rotational movement of the handle or knob is thus transmitted to actuate the mixing paddle 503.

When the cement is mixed, the user rotates the controllable portal assembly 507 to open the controllable port between the mixing chamber 502 and the dispensing syringe tube 535. The handle 543 is then turned to move the mixed material through the now open port into the syringe tube 535.

After the syringe tube 535 is filled, the user flips the hinged handle 543 across the center of the cap 547 to lock the handle in the second position (FIG. 39B) into a receiving slot 547A of the cap. This change of handle position releases the pressure of the crank handle cam 543 on the crank handle gear 548, permitting the spring biased gear to move upward, disengaging the gear from the paddle 503. In the second position, a hex cap 546 in the handle 543 engages the drive configuration 511A of a threaded plunger shaft 511. The paddle is thus disengaged so that it does not move or mix during the discharge of material from the apparatus, and the handle is engaged with threaded shaft 511 for driving the mixed material out through syringe tube 535.

The user then rotates handle 543 or the knobbed crank handle cap 547 to actuate the plunger mechanism (511, 518, 533) pushing the mixed material out the end of the syringe tube 535 and typically into a needle or cannula (not shown).

3.4 Component Structure

The needle housing 531 and needle housing cap 532 are preferably of the design shown and described in connection with FIG. 29. Advantageously the needle housing 531 has a detent bump 531A in the lower tube portion to hold the housing in place during shipment and preparation. The needle housing in place also aligns the vacuum port 501A with an opening 547B in the crank handle cap 547 to facilitate the attachment of a vacuum line (not shown). Alternatively, the vacuum port 501A can be disposed on the exterior of the mixing chamber or the end cap to facilitate attachment. Crank handle cap 547 houses the crank handle 543 and assembled knob components.

The crank handle is held in place by a crank handle cup comprising a pivot half 541 and a clamp half 542, each secured to the crank handle cap 547. The cup (541,542) also houses the crank handle gear 548, permitting it to slide longitudinally. The longitudinal position of the gear 548 is controlled by the cam surface 543A of the handle. The gear is loaded by spring 549 in the disengaged condition with respect to the mixing paddle 503. Thus when the handle is in the first position (FIG. 39A), the gear is engaged with the paddle. Pivoting the handle to the second position (FIG. 39B) releases the gear to its spring biased disengaged position.

The crank handle cup assembly (541, 542) also locks onto the top end of the paddle 503 to provide alignment between the paddle and the gear 548. The two halves 541, 542 are secured together as by screws 539 or snap-fit connections (not shown). The cup could also be molded as a one piece part.

The end cap 501 is advantageously similar to top cap 315 of FIG. 32. It attaches the upper cap assembly to the mixing chamber 502.

The mixing paddle 503 is advantageously similar to paddle 390 of FIG. 33, except that the drive end 503A has a gear configuration for interfacing with the crank handle gear 548. It is preferred that the top of the drive end 503A have lead-in edges to assist in engagement with the crank handle gear.

In other regards, the drive mechanisms for mixing and dispensing and the mechanisms for operating the controllable port between the mixing chamber and the dispensing chamber are the same as those described for other embodiments herein.

It can now be seen that the exemplary apparatus for manually mixing and dispensing components comprises a sealed mixing chamber having an access portal and a vacuum portal, a mixing unit in the mixing chamber to mix the components, and a first manually actuable drive mechanism associated with the mixing unit to actuate mixing.

The apparatus further includes a dispensing chamber connected to the sealed mixing chamber but which is isolated from the mixing chamber. A controllable portal is provided for opening a flow path between the sealed mixing chamber and the dispensing chamber after the components are mixed. A second manually actuable drive mechanism associated with the dispensing chamber is provided to drive the mixture from the dispensing chamber.

In advantageous forms, the mixing chamber is preloaded with bone cement powder. The first and second drive mechanisms comprise rotationally movable handles or knobs and preferably a common handle or knob. A mechanical switching arrangement can be provided to disengage the common handle or knob from the first drive mechanism. The preferred mixing unit comprises a mixing paddle, and the preferred second drive mechanism comprises a plunger shaft.

3.5 Biocompatible Fillers and Bone Cements for Filling Cavities

Suitable biocompatible bone void fillers are well known by those in the art and include, but are not limited to, bone cements, calcium phosphates, and bioresorbable polymers. Preferably, such fillers conform with the Food and Drug Administration's Class II Special Controls Guidance Document: Polymethylmethacrylate (PMMA) Bone Cement; Guidance for Industry and FDA.

In a preferred embodiment, the biocompatible filler is bone cement, more preferably, bone cement formed from a mixture of polymethylmethacrylate (PMMA), methyl methacrylate monomer, and a substance to radiopacify the cement mixture. The radiopacifier enables monitoring of the cement mixture using x-ray radiographs. Examples of radiopacifiers include barium salts, ceramic particles, or tantalum materials.

In an additional embodiment, the bone cement mixture can include one or more additional pharmaceutical agents, such as an antibiotic to reduce the risk of infection. One example of a suitable antibiotic for use in the bone cement mixture is tobramycin.

Suitable commercially available bone cement mixtures include, but are not limited to, Concert®, Concert® Cranioplast, Palacos, Palacos G, DePuy 1, 2, and 3, DePuy 1 with Gentamycin, Osteobond, Simplex-P, Simplex-P with Tobramycin, Versabond, Endurance, all commercially available, for example, commercially available from Advanced Biomaterial Systems, Inc., Chatham, N.J.

### 4.0 EXAMPLE

In an exemplary procedure, to correct a fractured vertebrae, the patient lies on an operating table, while the physician introduces a conventional spinal needle assembly into the soft tissue in the patient's back. The patient can lie facedown on the table, or on either side, or at an oblique angle, depending upon the physician's preference. Alternatively, the procedure can be performed through an open anterior procedure or an endoscopic anterior procedure.

The spinal needle assembly typically comprises a stylet slidably housed within a stylus. The assembly typically has, for example, about an 18 gauge diameter. Other gauge diameters can and will be used to accommodate appropriate guide pins, as will be described in greater detail below. For example, in certain instances, 13 gauge to 8 gauge needles may be suitable. In yet other applications, use of even larger needles than that may be desirable.

Under radiological, CT, or MRI monitoring, the physician advances the assembly through the soft tissue and into the targeted vertebra. The physician will typically administer a local anesthetic, for example, lidocaine, through the assembly. Other forms of anesthesia may be used.

The physician causes the spinal needle assembly to penetrate the cancellous bone of the targeted vertebrae. Access to the cancellous bone may be through the pedicle, known as transpedicular access. It is contemplated that posterolateral access, through the side of the vertebral body, may be preferred, if a compression fracture has collapsed the vertebral body below the plane of the pedicle, or for other reasons based upon the discretion and preference of the physician.

After positioning the spinal needle assembly in the cancellous bone, the physician holds the stylus and withdraws the stylet. Still holding the stylus, the physician slides a guide pin through the stylus and into the cancellous bone. The physician now removes the stylus, leaving the guide pin deployed within the cancellous bone.

Next, the physician makes a small incision in the patient's back sufficiently sized to accommodate a delivery tube device, such as a trocar or a cannula. The delivery tube device is inserted into the patient's vertebral body using a transpedicular or extrapedicular approach. A drill is inserted into the delivery tube device to create a channel in the vertebral body stopping just behind the anterior wall. The drill device is then removed from the delivery tube device.

The elastomer delivery device is inserted into the canal or tube of the delivery tube device. The elastomer delivery device is packed with the elastomer. A plunger is then made to apply a deforming force to the, elastomer thereby pushing the elastomer out of the end of the elastomer delivery device and into the vertebral body. The elastomer maintains its linear backbone structure until coming into contact with the bumper located on the guide rod. The deforming force created by the bumper causes the elastomer to expand outwardly. The combination of the deforming forces created by the plunger action and the bumper cause the elastomer to expand throughout the cancellous bone and create a cavity therein. Due to the application of stress or pressure, the elastomer material expands approximately 3 to 10 times its normal size.

Advantageously, the expanded elastomer material acts on the collapsed vertebral body to restore height in the collapsed vertebral body. Once the desired cavity is created, the action applied to the ratcheting device is reversed and the deforming force acting on the elastomer is removed. The removal of the deforming forces and the space created in the elastomer delivery device by the reversal of the plunger allows the elastomer to retract back into its linear backbone structure within the elastomer delivery device. Upon withdrawal of the elastomer from the cavity, the cavity creation device is removed from the delivery tube device. Next, the physician may introduce a bone cement mixture (described in detail in section above) or biomaterials into the newly formed cavity in the vertebral body. Preferably, delivery of the bone cement mixture is accomplished by way of a tube, hose, cannula, or needle. For example, by use of the Plexis™ system described above.

The above example is for explanatory purposes only and is not intended to limit the use of the device to the specific manner described.

From the above Summary, Description, Figures, and Example, it is clear that in one embodiment, a device for creating a cavity in mammalian tissue includes
(a) a holding sleeve, suitable for containing an elastomeric material, in operative communication with a bumper;
(b) a plunger for insertion into the holding sleeve,
wherein when the holding sleeve contains an elastomeric material and the plunger is inserted into the holding sleeve, operation of the plunger forces the elastomeric material against the bumper, the bumper configured to cause the elastomeric material forced against it to expand and create the cavity.

In another embodiment, a device for creating a cavity in mammalian tissue includes:
(a) a holding sleeve in operative communication with a bumper;
(b) the holding sleeve comprising an elastomeric material; and
(c) a plunger in operative communication with the holding sleeve,
wherein operation of the plunger forces the elastomeric material against the bumper causing the elastomeric material to expand.

In still another embodiment, a kit for assembly into a device for creating a cavity in mammalian tissue includes the following components:
(a) a bumper;
(b) a holding sleeve for operative communication with the bumper;
(c) an elastomeric material for insertion into the holding sleeve;
(d) a plunger suitable for insertion into the holding sleeve,
wherein when the components are assembled into the device and the elastomeric material is inserted into the tissue, operation of the plunger forces the elastomeric material against the bumper causing the elastomeric material to expand to create the cavity.

In still another embodiment, the a method of creating a cavity in mammalian tissue includes:
(a) inserting an elastomeric material into the tissue;
(b) applying a compressive force to the elastomer to expand the elastomer, thereby creating the cavity;
(c) contracting the elastomeric material to substantially its original dimensions; and
(d) removing the elastomeric material from the tissue.

In still yet another embodiment, a method of repairing, stabilizing, or reinforcing weak, damaged or diseased mammalian bone tissue with a device includes:
(a) inserting an elastomeric material into the tissue;
(b) applying a compressive force to the elastomer to expand the elastomer, thereby creating the cavity;
(c) contracting the elastomeric material to substantially its original dimensions;
(d) removing the elastomeric material from the tissue; and
(e) filling the cavity with a biocompatible filler.

Although various aspects have been described in considerable detail with reference to certain preferred embodiments and versions, other versions and embodiments are possible. Therefore, the spirit and scope of the appended claims should not be limited to the description of the versions and embodiments expressly disclosed herein.

1. An apparatus comprising:
a hollow sleeve having an open end;
a guide rod coupled to the hollow sleeve and extending axially from the open end;
a bumper coupled to a distal end of the guide rod;
a deformable material positionable between the open end and the bumper; and
a plunger adapted to move axially through the hollow sleeve to apply a compressive force on the deformable material against a counterforce applied by the bumper.

2. The apparatus of claim 1 wherein the deformable material is adapted to expand in response to the applied compressive force.

3. The apparatus of claim 1 wherein the deformable material comprises an elastomer.

4. The apparatus of claim 1 wherein the deformable material comprises a collapsible segmented elastomeric tube.

5. The apparatus of claim 1 further comprising an expandable casing having a first opening coupled to the open end of the hollow sleeve and a second open end coupled to the bumper.

6. The apparatus of claim 5 wherein the plunger is adapted to push the deformable material into the expandable casing by moving through the hollow sleeve.

7. The apparatus of claim 6 wherein the deformable material is bone cement.

8. A device for creating a cavity in mammalian tissue comprising:
(a) a holding sleeve, suitable for containing an elastomeric material, in co-axial communication with a bumper;
(b) a plunger for insertion into the holding sleeve,
wherein when the holding sleeve contains an elastomeric material and the plunger is inserted into the holding sleeve, operation of the plunger forces the elastomeric material against the bumper, the bumper configured to cause the elastomeric material forced against it to expand and create a cavity.

9. The device of claim 8, wherein the holding sleeve is adapted to carry the elastomeric material.

10. The device of claim 8, wherein the elastomeric material comprises an elastomer.

11. The device of claim 10, wherein the elastomer comprises a silicon elastomer.

12. The device of claim 8, wherein the elastomeric material is sterile.

13. The device of claim 8, wherein the elastomeric material comprises a collapsible segmented elastomeric tube.

14. The device of claim 13, wherein the collapsible segmented elastomeric tube comprises an elastomer.

15. The device of claim 9, wherein the elastomeric material comprises an elastomer in a compressible secondary casing.

16. The device of claim 15, wherein the compressible secondary casing is constructed of an elastomer.

17. The device of claim 8, wherein the holding sleeve comprises a guide rod and the guide rod is connected to the bumper.

18. The device of claim 1, wherein the bumper is pointed to facilitate insertion into the tissue.

19. The device of claim 1, further comprising a delivery tube device.

20. The device of claim 1, wherein the holding sleeve comprises female threads and the plunger comprises male threads, wherein the plunger is operated by rotating the plunger relative to the holding sleeve.

21. The device of claim 20, further comprising a ratcheted crank handle in communication with the plunger to rotate the plunger relative to the holding sleeve.

22. A kit for assembly into a device for creating a cavity in mammalian tissue comprising the following components:
a bumper;
a holding sleeve for operative communication with the bumper;
an elastomeric material for insertion into the holding sleeve;
a plunger suitable for insertion into the holding sleeve,
wherein when the components are assembled into the device and the elastomeric material is inserted into the tissue, operation of the plunger forces the elastomeric material against the bumper causing the elastomeric material to expand to create a cavity.

23. The kit of claim 22, wherein the holding sleeve comprises the elastomeric material.

24. The kit of claim 22, wherein the elastomeric material comprises an elastomer.

25. The kit of claim 24, wherein the elastomer comprises a silicon elastomer.

26. The kit of claim 22, wherein the elastomeric material comprises a collapsible segmented elastomeric tube.

27. The kit of claim 26, wherein the collapsible segmented elastomeric tube comprises an elastomer.

28. The kit of claim 22, further comprising a secondary elastomeric casing for encasing the elastomeric material.

29. The kit of claim 28, wherein the compressible secondary casing is constructed of an elastomer.

30. The kit of claim 22, wherein the holding sleeve comprises a guide rod and the guide rod is connected to the bumper.

31. The kit of claim 22, wherein the bumper is pointed to facilitate insertion into the tissue.

32. The kit of claim 22, further comprising a delivery tube device.

33. The kit of claim 22, wherein the holding sleeve comprises female threads and the plunger comprises male threads for operating the plunger by rotating the plunger relative to the holding sleeve.

34. The kit of claim 33, further comprising a ratcheted crank handle for rotating the plunger relative to the holding sleeve.

35. The kit of claim 22, further comprising a biocompatible filler.

36. The kit of claim 22, further comprising bone cement.

37. The kit of claim 22, further comprising calcium phosphate.

38. The kit of claim 22, further comprising a bioresorbable polymer.

39. The kit of claim 35, wherein the elastomeric material and biocompatible filler are sterile.

40. A method of creating a cavity in mammalian tissue comprising the steps of:
inserting an elastomeric material into a tissue;
expanding the elastomeric material by applying a compressive force to the elastomeric material against a bumper, the expanded elastomeric material creating a cavity;
contracting the elastomeric material to substantially its original dimensions; and
removing the elastomeric material from the tissue.

41. The method of claim 40, further comprising inserting the bumper into the tissue, wherein applying the compressive force comprises forcing the elastomeric material against the bumper.

42. The method of claim 40 wherein
inserting the elastomeric material into the tissue comprises:
inserting the elastomeric material into a holding sleeve that is coaxially coupled to the bumper; and
advancing a plunger through the holding sleeve.

43. The method of claim 40, wherein the elastomeric material comprises a collapsible segmented elastomeric tube and wherein expanding the elastomeric material comprises applying a compressive force to the collapsible segmented elastomeric tube and causing elastomeric portions of the collapsible segmented elastomeric tube to expand.

44. The method of claim 40, wherein the elastomeric material comprises an elastomer in a compressible secondary casing and wherein expanding the elastomeric material comprises pushing the elastomeric material into the compressible secondary casing.

45. The method of claim 44, wherein the compressible secondary casing is constructed of an elastomer and wherein pushing the elastomeric material into the compressible secondary casing causes the compressible secondary casing to expand.

46. The method of claim 42, wherein the holding sleeve comprises a guide rod and the guide rod is connected to the bumper.

47. The method of claim 40, further comprising utilizing a pointed end of the bumper to insert a holding sleeve containing the elastomeric material into the tissue.

48. The method of claim 40 further comprising inserting a delivery tube device.

49. The method of claim 42, wherein advancing a plunger through the holding sleeve comprises rotating the plunger relative to the holding sleeve.

50. The method of claim 42, wherein advancing a plunger through holding sleeve further comprises rotating a ratcheted crank handle relative to the holding sleeve.

51. The method of claim 40 further comprising filling the cavity with a biocompatible filler.

52. The method of claim 51, wherein filling the cavity with a biocompatible filler comprises adding the biocompatible filler by way of the delivery tube device.

53. A device for creating and filling a cavity in a mammalian body comprising:
an apparatus for dispensing a biocompatible filler; and
an elastomeric casing attached to the apparatus,
wherein when the biocompatible filler is forced into the elastomeric casing by the apparatus, the elastomeric casing expands thereby creating and filling the cavity.

54. The device of claim 53, wherein the elastomeric casing is biocompatible.

55. The device of claim 53, wherein the elastomeric casing and biocompatible filler are implanted in a mammalian body.

56. The device of claim 53, wherein the elastomeric casing comprises an elastomer.

57. The device of claim 53, wherein the biocompatible filler comprises bone cement.

58. The device of claim 53, wherein the biocompatible filler comprises calcium phosphate.

59. The device of claim 53, wherein the biocompatible filler comprises a bioresorbable polymer.

60. A method of creating and filling a cavity in mammalian tissue comprising the steps of:
inserting an elastomeric casing in the tissue; and
filling the elastomeric casing with a biocompatible filler to expand the elastomeric casing, thereby creating and filling the cavity.

61. The method of claim 60, wherein the elastomeric casing is biocompatible.

62. The method of claim 60, further comprising implanting the elastomer casing filled with the biocompatible filler in the mammalian tissue.

63. The method of claim 60, wherein the elastomeric casing comprises an elastomer.

64. The method of claim 60, wherein the biocompatible filler comprises bone cement.

65. The method of claim 60, wherein the biocompatible filler comprises calcium phosphate.

66. The method of claim 60, wherein the biocompatible filler comprises a bioresorbable polymer.

67. A kit for assembly into a device for creating and filling a cavity in mammalian tissue comprising the following components:
an elastomeric casing;
a biocompatible filler for filling the elastomeric casing,
an apparatus for filling the elastomer casing,
wherein when the components are assembled into the device and the elastomeric casing is inserted into the tissue, the biocompatible filler fills the elastomeric casing causing the elastomeric casing to expand to create and fill a cavity.

68. The kit of claim 67, wherein the elastomeric casing is biocompatible.

69. The kit of claim 67, wherein the biocompatible elastomeric casing and biocompatible filler are implanted in a mammalian body.

70. The kit of claim 67, wherein the biocompatible elastomeric casing comprises an elastomer.

71. The kit of claim 67, wherein the biocompatible filler comprises bone cement.

72. The kit of claim 67, wherein the biocompatible filler comprises calcium phosphate.

73. The kit of claim 67, wherein the biocompatible filler comprises a bioresorbable polymer.

74. An implant for filling a cavity in mammalian tissue comprising a biocompatible elastomeric casing adapted to contain a biocompatible filler.

75. The implant of claim 74, wherein the biocompatible elastomeric casing comprises an elastomer.

76. The implant of claim 74, wherein the biocompatible filler comprises bone cement.

77. The implant of claim 74, wherein the biocompatible filler comprises calcium phosphate.

78. The implant of claim 74, wherein the biocompatible filler comprises a bioresorbable polymer.

79. The implant of claim 74, wherein the implant is implanted in a mammalian body.
